(19) United States
(12) Patent Application Publication
(10) Pub. No.: US 2025/0327077 A1
(43) Pub. Date: Oct. 23, 2025

(54) ANTISENSE MODULATION OF SETBP1 EXPRESSION

(71) Applicant: THE ROYAL INSTITUTION FOR THE ADVANCEMENT OF LEARNING/MCGILL UNIVERSITY, Montreal (CA)

(72) Inventors: Carl ERNST, Montreal (CA); Lilit ANTONYAN, Montreal (CA)

(21) Appl. No.: 19/184,510

(22) Filed: Apr. 21, 2025

Related U.S. Application Data
(60) Provisional application No. 63/637,053, filed on Apr. 22, 2024.

(51) Int. Cl. C12N 15/113; A61P 35/00

(52) U.S. Cl. CPC C12N 15/1135; A61P 35/00; C12N 2310/11; C12N 2310/14; C12N 2310/315; C12N 2310/321; C12N 2310/33; C12N 2320/34

(57) ABSTRACT

Described herein are reagents, products, methods, and uses for modulating SETBP1 expression, and uses thereof, such as for inhibiting SETBP1 expression in a cell, and for the treatment of SETBP1-associated diseases, such as Schinzel-Giedion Syndrome and SETBP1-associated cancer. The products include antisense oligonucleotides, such as modified antisense oligonucleotides, that target a region of the SETBP1 gene.

### CROSS REFERENCE TO RELATED APPLICATIONS

This application claims the benefit of U.S. Provisional Application Ser. No. 63/637,053, filed on Apr. 22, 2024, which incorporated herein by reference in its entirety.

### SEQUENCE LISTING

This application contains a Sequence Listing in electronic format entitled G11168-00504_SeqList.xml, created on Apr. 21, 2025 and having a size of 122,000 bytes. The information in the electronic format of the sequence listing is incorporated herein by reference in its entirety.

### FIELD OF THE DISCLOSURE

The present disclosure generally relates to oligonucleotides and uses thereof. More specifically, the present disclosure is concerned with products, methods and uses for inhibiting SETBP1 expression, and uses thereof, such as for the prevention or treatment of SETBP1-associated diseases, such as Schinzel-Giedion Syndrome and SETBP1-associated cancer.

### BACKGROUND OF THE DISCLOSURE

Schinzel-Giedion Syndrome (SGS) is a severe neurodevelopmental and multisystemic disease caused by germline heterozygous missense mutations in a mutational hotspot found in SETBP1, leading to increased protein stability, classifying this disease as gain-of-function (GOF) [1-4]. This hotspot is found within the degron motif located in the SKI-homology domain [2] of SETBP1, which is targeted by a β-TrCP E3 ubiquitin ligase [2, 5]. Hotspot mutations decrease the ability of the E3 ligase to recognize the degron motif in SETBP1, leading to increased protein stability and accumulation of SETBP1 in the cell [2]. Symptoms of SGS are severe developmental and growth delay, progressive brain atrophy, delayed myelination, progressive atrophy of white matter, distorted neuronal layering, hydronephrosis, midface retraction, severe seizures, neuroepithelial tumors [6], bone abnormalities and other congenital malformations [4, 7, 8]. Neoplastic tumors can also occur around the spinal cord [9]. The symptoms are so severe that children suffering from SGS die early in life [6]. There is no known cure for either one of these diseases and treatment is symptom based. Furthermore, somatic mutations in SETBP1 in the identical hotspot region as for SGS are oncogenic driver mutations causative of chronic myeloid leukemia (CML) [4], identified through cancer cell sequencing. This is distinguished from SGS germline mutations in SETBP1.

Increased expression of SETBP1 has also been observed in certain cancers, such as hematological cancers, including acute myeloid leukemia (AML), chronic myelomonocytic leukemia (CMML), myelodysplastic syndrome (MDS), and solid tumors, such as bladder cancer [12].

There is thus a need for new approaches for the treatment of SETBP1-associated diseases, such as SGS and SETBP1-associated cancer.

The present description refers to a number of documents, the content of which is herein incorporated by reference in their entirety.

### SUMMARY OF THE DISCLOSURE

The present disclosure generally relates to oligonucleotides and uses thereof. More specifically, the present disclosure is concerned with products (such as antisense oligonucleotides, methods and uses for modulating SETBP1 expression, and uses thereof, such as for inhibiting SETBP1 expression in a cell and for the prevention or treatment of SETBP1-associated diseases (such as Schinzel-Giedion Syndrome and SETBP1-associated cancer), or the prevention, treatment, delay of onset, reversal of progression, and/or reduction of severity of one or more symptoms of such diseases.

In various aspects and embodiments, the present disclosure provides the following items:
1. An antisense oligonucleotide (ASO) comprising a sequence that is at least 80% complementary to a target sequence within the SETBP1 nucleic acid sequence of SEQ ID NO: 20.
2. The ASO of item 1, comprising a sequence that is at least 90% complementary to a target sequence within the SETBP1 nucleic acid sequence of SEQ ID NO: 20.
3. The ASO of item 1 or 2, comprising a sequence that is 100% complementary to a target sequence within the SETBP1 nucleic acid sequence of SEQ ID NO: 20.
4. The ASO of any one of items 1-3, wherein the target sequence comprises at least 5, 6, 7, 8, or 9 contiguous nucleotides of SEQ ID NO: 20.
5. The ASO of any one of items 1-4, wherein the target sequence comprises at least 10, 11, 12, 13, 14 or 15 contiguous nucleotides of SEQ ID NO: 20.
6. The ASO of any one of items 1-5, which is 10-30 nucleotides in length.
7. The ASO of any one of items 1-6, which is 15-25 nucleotides in length.
8. The ASO of any one of items 1-7, wherein the target sequence is within a coding sequence of the SETBP1 nucleic acid sequence of SEQ ID NO: 20.
9. The ASO of any one of items 1-8, wherein the target sequence is within exon 2, 4 or 6 of the SETBP1 nucleic acid sequence of SEQ ID NO: 20.
10. The ASO of any one of items 1-8, wherein the target sequence is within exon 2 of the SETBP1 nucleic acid sequence of SEQ ID NO: 20.
11. The ASO of any one of items 1-8, wherein the target sequence is within exon 4 of the SETBP1 nucleic acid sequence of SEQ ID NO: 20.
12. The ASO of any one of items 1-8, wherein the target sequence is within exon 6 of the SETBP1 nucleic acid sequence of SEQ ID NO: 20.
13. The ASO of any one of items 1-12, wherein the target sequence is outside the region encoding the SKI-homology domain of SETBP1.
14. The ASO of any one of items 1-13, wherein the target sequence is within a region of SEQ ID NO: 20, wherein the region is defined by a 5′ end up to 100 nucleotides 5′ from the nucleotide sequence of one or more of SEQ ID NOs: 7-19 and a 3′ end up to 100 nucleotides 3′ from the nucleotide sequence of one or more of SEQ ID NOs: 7-19 and 63-74.
15. The ASO of any one of items 1-14, wherein the target sequence is within a region of SEQ ID NO: 20, wherein the region is defined by a 5′ end up to 75 nucleotides 5′ from the nucleotide sequence of one or more of SEQ ID NOs: 7-19 and a 3′ end up to 75 nucleotides 3′ from the nucleotide sequence of one or more of SEQ ID NOs: 7-19 and 63-74.
16. The ASO of any one of items 1-15, wherein the target sequence is within a region of SEQ ID NO: 20, wherein the region is defined by a 5′ end up to 50 nucleotides 5′ from the nucleotide sequence of one or more of SEQ ID NOs: 7-19 and a 3′ end up to 50 nucleotides 3′ from the nucleotide sequence of one or more of SEQ ID NOs: 7-19 and 63-74.
17. The ASO of any one of items 1-16, wherein the target sequence is within a region defined by the nucleotide sequence of one or more of SEQ ID NOs: 63-74.
18. The ASO of any one of items 1-17, comprising one or more modifications (a modified ASO).
19. The ASO of item 18, wherein the modified ASO comprises one or more modified internucleoside linkages, and/or one or more modified sugars, and/or one or more modified nucleobases.
20. The ASO of item 19, wherein the one or more modified internucleoside linkages comprise one or more of phosphorothioate, phosphorodithioate, phosphoramidate (e.g., dimethylaminophosphoramidate), the phosphonocarboxylate (e.g., phosphonoacetate), thiophosphonocarboxylate (e.g., thiophosphonoacetate), alkylphosphonate (e.g., methylphosphonate, boranophosphonate, and phosphorodithioate).
21. The ASO of anyone of items 18-20, wherein the one or more modifications comprise one or more nucleotide sugar modifications, such as one or more 2′ sugar modifications.
22. The ASO of item 21, wherein the one or more nucleotide sugar modifications comprise one or more of 2′-O-alkyl (e.g., 2′-O-methyl), 2′-deoxy, 2′-O-alkyl-O-alkyl (e.g., 2′-methoxyethyl (2′-MOE)), 2′-fluoro, 2′-amino, 2′-arabinosyl nucleotide, 2′-F-arabinosyl nucleotide, locked nucleic acid (LNA) nucleotide, 2′-amido bridge nucleic acid, unlocked nucleic acid (ULNA) nucleotide, 4′-thioribosyl nucleotide, constrained ethyl (cET), arabino nucleic acid (ANA), 2′-fluoro-ANA (FANA), or thiomorpholino.
23. The ASO of any one of items 1-122, comprising a gapmer structure comprising an internal gap region flanked by 5′ and 3′ wing regions, wherein the internal and wing regions differ at least by nucleoside type and/or internucleoside linkage type.
24. The ASO of item 23, wherein the internal gap region comprises DNA nucleosides and the flanking wing regions comprise RNA nucleosides.
25. The ASO of item 23 or 24, wherein the flanking wing regions comprise 2′-MOE and/or LNA bases.
26. The ASO of any one of items 23-25, wherein each of the internal gap region and flanking wing regions comprises 5-10 nucleosides.
27. The ASO of any one of items 1-26, wherein the target sequence is set forth in Table 1, and/or wherein the ASO is set forth in Table 1 or 2.
28. A composition comprising the ASO of any one of items 1-27 and a pharmaceutically acceptable carrier.
29. A method of inhibiting SETBP1 expression in a cell, comprising contacting the cell with the ASO of any one of items 1-27 or the composition of item 28.
30. The method of item 29, which is an in vitro method.
31. The method of item 29, which is an in vivo method and the cell is in an animal.
32. The method of item 31, wherein the animal is a human.
33. A method for treating or preventing a disease in a subject, comprising administering a therapeutically or prophylactically effective amount of the ASO of any one of items 1 to 27, or the pharmaceutical composition of item 27 to a subject suffering from or susceptible to the disease.
34. The method of item 33, wherein the disease is a SETBP1-associated disease.
35. The method of item 33 or 34, wherein the disease is Schinzel-Giedion Syndrome or SETBP1-associated cancer.
36. The method of any one of items 33-35, wherein the subject is human.
37. The ASO of any one of items 1-27 or the pharmaceutical composition of item 28 for use in inhibiting SETBP1 expression in a cell.
38. The ASO of any one of items 1-27 or the pharmaceutical composition of item 28 for use as a medicament.
39. The ASO of any one of items 1-27 or the pharmaceutical composition of item 28 for use in treating or preventing a SETBP1-associated disease in a subject.
40. The ASO or pharmaceutical composition for use of item 39, wherein the disease is Schinzel-Giedion Syndrome or SETBP1-associated cancer.
41. The ASO or pharmaceutical composition for use of item 39 or 40, wherein the subject is human.
42. Use of the ASO of any one of items 1-27 or the pharmaceutical composition of item 28 for inhibiting SETBP1 expression in a cell.
43. Use of the ASO of any one of items 1-27 for the preparation of a composition for inhibiting SETBP1 expression in a cell.
44. Use of the ASO of any one of items 1-27 or the pharmaceutical composition of item 28 as a medicament.
45. Use of the ASO of any one of items 1-27 or the pharmaceutical composition of item 28 for treating or preventing a SETBP1-associated disease in a subject.
46. Use of the ASO of any one of items 1-27 or the pharmaceutical composition of item 28 for the preparation of a medicament for treating or preventing a SETBP1-associated disease in a subject.
47. The use of item 45 or 46, wherein the disease is Schinzel-Giedion Syndrome or SETBP1-associated cancer.
48. The use of any one of items 45-47, wherein the subject is human.

Other objects, advantages, and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-1B: SETBP1's degron motif and recognition by the SCR-β-TrCP1 E3 ubiquitin ligase is altered by SGS mutations affecting protein degradation. A. Diagram demonstrating SETBP1's degron recognition by the SCR-β-TrCP1 E3 ubiquitin ligase, leading to its degradation by the proteasome. Degron sequence is indicated inside the blue square. Ser869 and Thr873 must be phosphorylated to be recognized by the E3 ubiquitin ligase. Sequence shown corresponds to SEQ ID NO: 75. B. Mutations leading to the respective amino acid changes indicated below affect E3 ubiquitin ligase recognition of SETBP1's degron, affecting SETBP1 degradation. Sequence shown corresponds to SEQ ID NO: 76.

FIG. 2: SETBP1 levels are increased in SGS NPCs. Western blot and quantification of SETBP1 from lysates obtained from SGS patients and respective controls. Protein quantification of data from n=3 cell lines with 3 independent experiments performed, Student's t-test, **p<0.01, error bars denote SEM.

FIGS. 3A-3B: Half-life assessment of SETBP1. A. CHX treatment of control NPCs for up to 6 hrs. B. CHX treatment of SGS NPCs for up to 144 hours. Protein quantification of data from n=3 cell lines with 3 independent experiments performed, Two-way ANOVA followed by Tukey's multiple comparison test, *p<0.05, **p<0.01, error bars denote SEM.

FIGS. 4A-4H: ASOs targeting SETBP1 to reduce its expression in control and SGS cell lines. A. ASO screen with 8 different ASOs targeting different regions of SETBP1. B. Chemical structure of PS/PD ASO gapmer. ASO3 and ASO7 are highlighted with a red arrow. C. 6-FAM fluorescent ASO transfected into NPC demonstrates efficient transfection efficiency. D. WB and E. quantification of SETBP1 following transfection of respective ASOs at 100 nM for 2 days. Protein quantification of data from n=3 cell lines with 1 independent experiment performed, One-way ANOVA test performed. F. WB and G. quantification of SETBP1 following ASO treatment at increasing doses in a control line and 4 different SGS lines. Protein quantification of data from n=4 cell lines with 2 independent experiments performed, One-way ANOVA followed by Dunnett's multiple comparison test, *p<0.05, error bars denote SEM. H. Quantification of relative SETBP1 mRNA expression from lysates assayed in FIG. 4F, One-way ANOVA.

FIGS. 5A-5D: 2′MOE/PS ASOs targeting SETBP1 to reduce its expression in SGS and control NPCs lines. A. Tiled ASO versions of ASO3 and ASO7. B. Chemical structure of 2′MOE/PS ASO gapmer. C. WB and D. quantification of SETBP1 following ASO treatment at 50 nM for 2 days. Protein quantification of data from n=4 cell lines with 3 independent experiments performed, One-way ANOVA followed by Dunnett's multiple comparison test, *p<0.05, error bars denote SEM.

FIGS. 6A-6H: ASOs targeting SETBP1 to reduce its expression other disease models. A. Cartoon of HEK293-G870S-SETBP1 cells. B. WB and C. quantification of SETBP1 following 2-day ASO treatment at 75 nM and 100 nM in G870S-SETBP1 HEK293 lines. Protein quantification of data from n=1 cell line with 3 independent experiments performed, One-way ANOVA followed by Dunnett's multiple comparison test or Student's T-test, *p<0.05, **p<0.01, ***p<0.001, ****p<0.0001, error bars denote SEM. D. Cartoon of SGS neuroepithelial tumour cells. E. WB and F. quantification of SETBP1 following 2-day ASO treatment in SGS tumour cell lines. Protein quantification of data from n=1 cell line with 3 independent experiments performed, One-way ANOVA followed by Dunnett's multiple comparison test or Student's T-test, *p<0.05, **p<0.01, error bars denote SEM. G. Brightfield images of SGS tumour cells after ASO transfection at 50 nM for 2 days and H. 4 days.

FIGS. 7A-7E: SETBP1 mRNA is knocked-down following treatment with ASO3-MOE. A. QPCR of SETBP1 following treatment with increasing doses of ASO3-MOE and ASO3scr-MOE in 3 SGS NPC lines. Quantification of data from n=3 cell lines with 2 replicates performed per dose, Student's t-test for each ASO3-MOE and ASO3scr-MOE dose pair, error bars denote SD. B. QPCR of SETBP1 following treatment with increasing doses of ASO3-MOE and ASO3scr-MOE in a WT marmoset NPC line. Quantification of data from n=1 cell line with 2 replicates performed per dose, Student's t-test for each ASO3-MOE and ASO3scr-MOE dose pair, error bars denote SD. C. SETBP1 mRNA downregulation data from RNAseq assessment (normalized and log 1p transformed). Quantification of data from n=3 cell lines with 1 replicate performed per dose, Student t-tests for each ASO3-MOE and ASO3scr-MOE dose pair, error bars denote SD. D. SETBP1 expression normalized and log 1p transformed in marmoset and pooled SGS NPCs in ASO3-MOE and ASO3scr-MOE treated samples. E. SETBP1 expression normalized and log 1p transformed in marmoset and individual SGS NPC cell lines in ASO3-MOE and ASO3scr-MOE treated samples.

FIGS. 8A-8C: ASO3 downregulates expression of HA-SETBP1 in HEK293 cells. A. Cartoon of HEK293 cells overexpressing HA-SETBP1. B. Western blot of HA-SETBP1 treated with 100 nM and 200 nM ASO3. C. Quantification of SETBP1 protein downregulation WB data following administration with ASO3-MOE at 100 and 200 nM, n=1 cell line with 3 independent experiments performed, One-way ANOVA, error bars denote SD.

FIGS. 9A-9E: ASO3 potency and downregulation of SETBP1 mRNA expression in neurons. A. Cartoon of NPCs. B. IC₅₀ curve of ASO3-MOE calculated following transfection of ASO3-MOE into 3 SGS NPC lines and qPCR analysis of SETBP1 expression. C. Cartoon of forebrain neurons, D. ICC of forebrain neurons stained with TUJ1 and MAP2. Scale bar represents 1 mm. E. QPCR of SETBP1 following treatment with 300 nM of ASO3-MOE and ASO3scr-MOE in n=4 cell SGS forebrain neuron lines, Student's t-test, error bars denote SD.

FIGS. 10A-10B: Effect of ASO treatment on WT mice. A. Study design for post-natal ASO assessment. B. Graph assessing weekly weight of control mice following ASO injection.

FIGS. 11A-11E: Human SETBP1 exon sequences. Exons 1-6 correspond to SEQ ID NOs 1-6. Coding sequence shown in bold (SEQ ID NO: 21). Target sequences corresponding to ASO1, ASO2, ASO3, ASO4, ASO5, ASO6, ASO7, and ASO8 (SEQ ID NOs: 7-14, respectively) are highlighted. For target sequences of ASO2 and ASO3, which overlap, the ASO2 target sequence is italicized, and the ASO3 target sequence is underlined. Target sequence for ASO3.1 is GAAAGAGGAAACACAAACCGCAGGC (SEQ ID NO: 15); target sequence for ASO3.2 is CACAAACCGCAGGCCCCCGC (SEQ ID NO: 16); target sequence for ASO3.3 is AGGCGGAAAGAGGAAACACAA (SEQ ID NO: 17); target sequence for ASO7.1 is ATACTTTCCAACTCTGAGGG (SEQ ID NO: 18); target sequence for ASO7.2 is TTAACAGGATACTTTCCAAC (SEQ ID NO: 19). Full sequence containing exons 1-6 corresponds to SEQ ID NO: 20.

FIGS. 12A-12B: Human SETBP1 coding sequence (SEQ ID NO: 21). GenBank accession NM_015559; features shown in FIG. 12. Target sequences corresponding to ASO 8 (positions 1-20; SEQ ID NO: 14), ASO1 (positions 176-195; SEQ ID NO: 7), ASO5 (positions 782-801; SEQ ID NO: 11), ASO4 (positions 840-859; SEQ ID NO: 10), ASO6 (positions 1271-1290; SEQ ID NO: 12), ASO7 (positions 1347-1366; SEQ ID NO: 13), ASO2 (positions 4650-4669; SEQ ID NO: 8), and ASO3 (positions 4653-4673; SEQ ID NO: 9 are highlighted, with the ASO2 target sequence italicized and the ASO3 target sequence underlined. Nucleotides 2116-2751 (SEQ ID NO: 61) define the SKI-homology domain.

FIG. 13: Human SETBP1 amino acid sequence (SEQ ID NO: 22). Amino acids 706-917 (SEQ ID NO: 62) define the SKI-homology domain.

FIGS. 14A-14C: Details of human SETBP1 in GenBank accession NM_015559.

### DETAILED DESCRIPTION OF ILLUSTRATIVE EMBODIMENTS

Described herein are strategies for inhibiting of SETBP1 expression, including antisense oligonucleotide (ASO)-based inhibition, which may be used for example for preventing or treating SETBP1-associated diseases, such as Schinzel-Giedion Syndrome and SETBP1-associated cancer.

Antisense oligonucleotides (ASOs) are short stretches of DNA that bind to complementary mRNAs, creating a complex that can be recognized by endogenous RNase H and degraded, or that can block translation or splicing by steric hindrance [10, 11]. Therefore, ASOs can efficiently decrease mRNA levels and protein levels by targeting mRNA. ASOs have shown to have the ability to enter the CNS after intrathecal delivery, distributing widely through the brain [10] and are taken up by neurons [10].

In an aspect, the present disclosure provides an antisense oligonucleotide (ASO) that is capable of hybridizing with a SETBP1 nucleic acid or a region thereof, and is capable of inhibiting SETBP1 expression (to reduce or inhibit the production and levels of SETBP1 protein). In an embodiment, there is provided an ASO comprising a sequence that is at least 70% complementary, in further embodiments at least 75%, 80%, 85%, 90%, 91%, 92%, 93, 94%, or 95% to a target sequence within a SETBP1 nucleic acid sequence, such as the SETBP1 nucleic acid sequence of SEQ ID NO: 20. In an embodiment, there is provided an ASO that is 100% complementary to a target sequence within the SETBP1 nucleic acid sequence of SEQ ID NO: 20.

In embodiments, the ASO is at least 5, 10, 15 or 20 nucleotides in length. In embodiments, the ASO is not more than 20, 25, 30, 35, 40, 45, 50, 55, 60, 65, 70 or 75 nucleotides in length. In embodiments the ASO is 10-30 or 15-25 nucleotides in length. In embodiments the ASO is 20-25, 20 or 25 nucleotides in length.

In embodiments, the target sequence is within an exon of SETBP1 (e.g., exon 2, 4 or 6). In embodiments the target sequence is within a coding sequence of SETBP1. In an embodiment, the target sequence is outside the region encoding the SKI-homology domain of SETBP1.

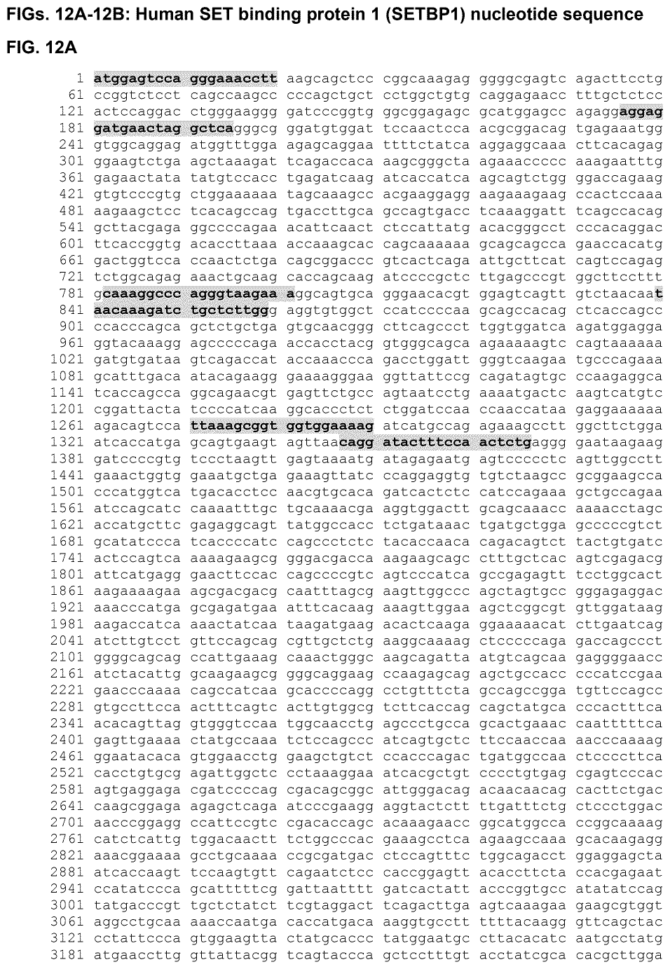

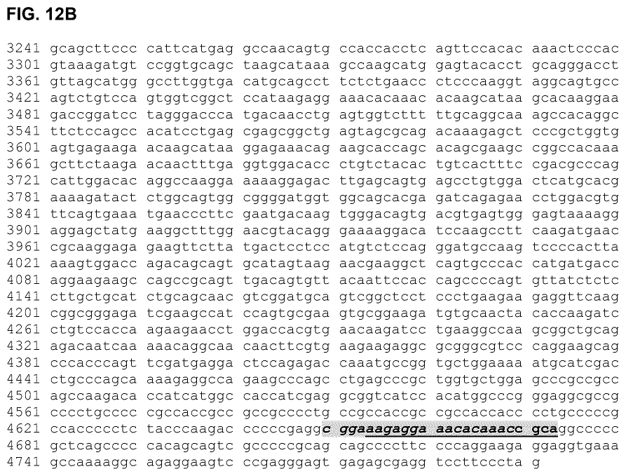

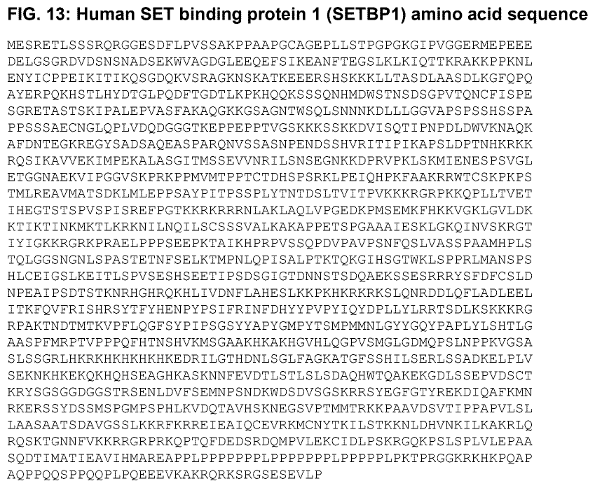

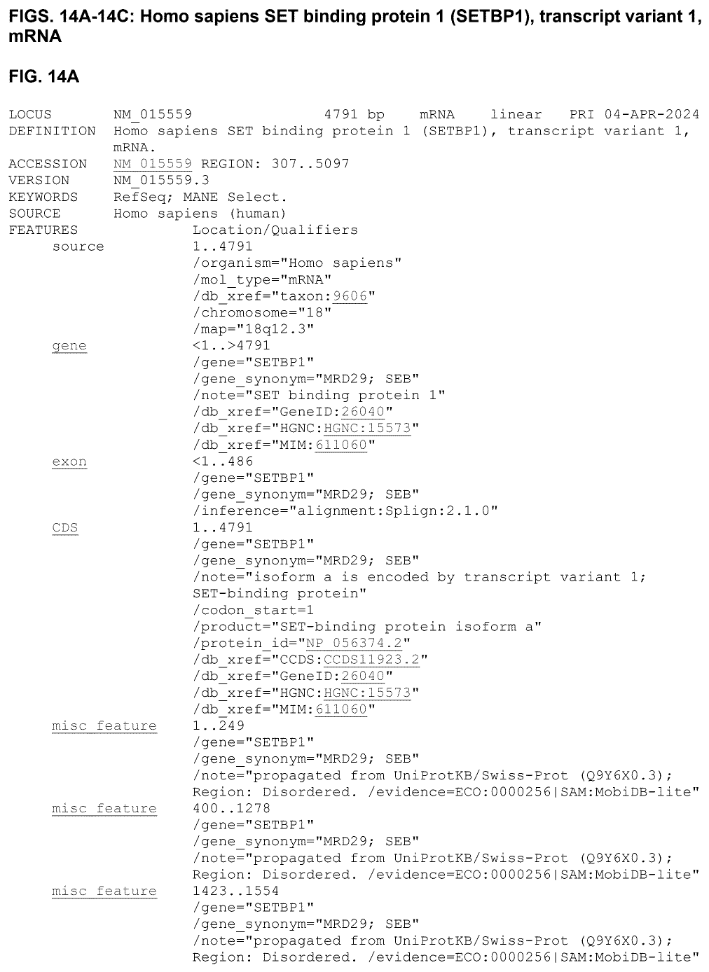

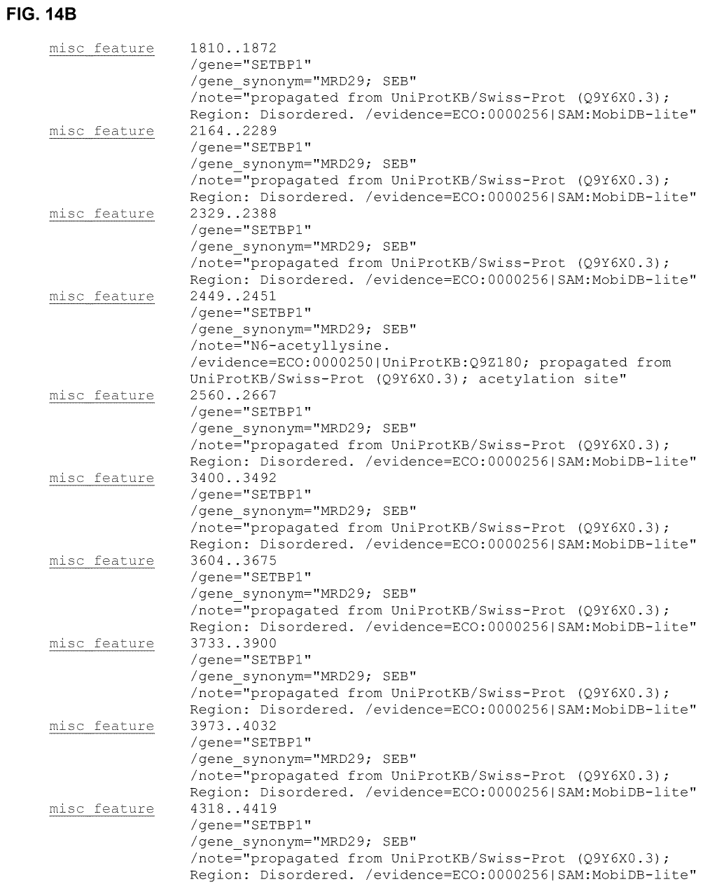

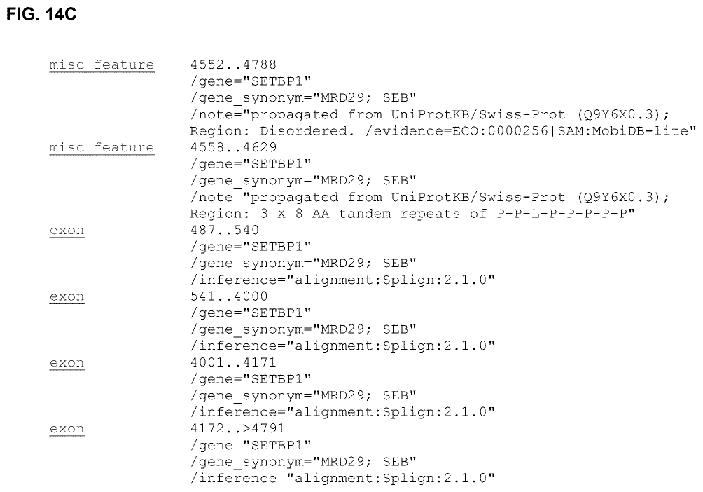

In embodiments, the target sequence is within a region of SETBP1 defined by positions 1-1366 (SEQ ID NO: 63) of the SETBP1 coding sequence set forth in FIG. 12 (SEQ ID NO: 21). In embodiments, the target sequence is within a region of SETBP1 defined by positions 1-1370 (SEQ ID NO: 64) of the SETBP1 coding sequence set forth in FIG. 12 (SEQ ID NO: 21). In embodiments, the target sequence is within a region of SETBP1 defined by positions 1-859 (SEQ ID NO: 65) of the SETBP1 coding sequence set forth in FIG. 12 (SEQ ID NO: 21). In embodiments, the target sequence is within a region of SETBP1 defined by positions 782-1366 (SEQ ID NO: 66) of the SETBP1 coding sequence set forth in FIG. 12 (SEQ ID NO: 21). In embodiments, the target sequence is within a region of SETBP1 defined by positions 782-1370 (SEQ ID NO: 67) of the SETBP1 coding sequence set forth in FIG. 12 (SEQ ID NO: 21). In embodiments, the target sequence is within a region of SETBP1 defined by positions 1-195 (SEQ ID NO: 68) of the SETBP1 coding sequence set forth in FIG. 12 (SEQ ID NO: 21). In embodiments, the target sequence is within a region of SETBP1 defined by positions 782-859 (SEQ ID NO: 69) of the SETBP1 coding sequence set forth in FIG. 12 (SEQ ID NO: 21). In embodiments, the target sequence is within a region of SETBP1 defined by positions 1271-1366 (SEQ ID NO: 70) of the SETBP1 coding sequence set forth in FIG. 12 (SEQ ID NO: 21). In embodiments, the target sequence is within a region of SETBP1 defined by positions 1271-1370 (SEQ ID NO: 71) of the SETBP1 coding sequence set forth in FIG. 12 (SEQ ID NO: 21). In embodiments, the target sequence is within a region of SETBP1 defined by positions 1343-1370 (SEQ ID NO: 72) of the SETBP1 coding sequence set forth in FIG. 12 (SEQ ID NO: 21). In embodiments, the target sequence is within a region of SETBP1 defined by positions 4647-4673 (SEQ ID NO: 73) of the SETBP1 coding sequence set forth in FIG. 12 (SEQ ID NO: 21). In embodiments, the target sequence is within a region of SETBP1 defined by positions 4647-4682 (SEQ ID NO: 74) of the SETBP1 coding sequence set forth in FIG. 12 (SEQ ID NO: 21). In embodiments, the target sequence is within a region of SETBP1 defined by a starting position of 4647, 4652, 4654 or 4663 and an end position of 4667, 4673, 4676 or 4682 of the SETBP1 coding sequence set forth in FIG. 12 (SEQ ID NO: 21). In embodiments, the target sequence is within a region of SETBP1 defined by a starting position of 1343, 1347 or 1351 and an end position of 1362, 13667 or 1370 of the SETBP1 coding sequence set forth in FIG. 12 (SEQ ID NO: 21).

In embodiments, the target sequence is within a region of SEQ ID NO: 20 or 21 wherein the region is defined by a 5′ end up to 100 nucleotides 5′ from the nucleotide sequence of one or more of SEQ ID NOs: 63-74 and a 3′ end up to 100 nucleotides 3′ from the nucleotide sequence of one or more of SEQ ID NOs: 63-74. In embodiments, the region is defined by a 5′ end up to 75, up to 50, or up to 25 nucleotides from the nucleotide sequence of one or more of SEQ ID NOs: 63-74 and a 3′ end up to 75, up to 50, or up to 25 nucleotides 3′ from the nucleotide sequence of one or more of SEQ ID NOs: 63-74.

In embodiments, the target sequence is within a region of SEQ ID NO: 20 or 21 wherein the region is defined by a 5′ end up to 100 nucleotides 5′ from the nucleotide sequence of one or more of SEQ ID NOs: 7-19 and a 3′ end up to 100 nucleotides 3′ from the nucleotide sequence of one or more of SEQ ID NOs: 7-19. In embodiments, the region is defined by a 5′ end up to 75, up to 50, or up to 25 nucleotides from the nucleotide sequence of one or more of SEQ ID NOs: 7-19 and a 3′ end up to 75, up to 50, or up to 25 nucleotides 3′ from the nucleotide sequence of one or more of SEQ ID NOs: 7-19.

In embodiments, an ASO described herein is a non-naturally occurring nucleic acid. In embodiments, an ASO described herein is a modified ASO, i.e., comprising one or more modifications. In embodiments, such one or more modifications comprise one or more modified internucleoside linkages, and/or one or more sugar modifications (such as one or more 2′ sugar modifications), and/or or one or more modified nucleobases (e.g., 5-methyl cytosine).

In embodiments, the one or more modified internucleoside linkages comprise one or more of phosphorothioate, phosphorodithioate, phosphoramidate (e.g., dimethylaminophosphoramidate), phosphonocarboxylate (e.g., phosphonoacetate), thiophosphonocarboxylate (e.g., thiophosphonoacetate), alkylphosphonate (e.g., methylphosphonate, boranophosphonate, and phosphorodithioate.

In embodiments, at least 20, 30, 40, 50 60, 70 80 or 90% of the internucleoside linkages in the ASO are modified internucleoside linkages.

In embodiments, the one or more nucleotide sugar modifications comprise one or more of 2′-O-alkyl (e.g., 2′-O-methyl), 2′-O-alkyl-O-alkyl (e.g., 2′-methoxyethyl (2′-MOE)), 2′-fluoro, 2′-amino, 2′-arabinosyl nucleotide, 2′-F-arabinosyl nucleotide, locked nucleic acid (LNA) nucleotide, 2′-amido bridge nucleic acid, unlocked nucleic acid (ULNA) nucleotide, 4′-thioribosyl nucleotide, constrained ethyl (cET), arabino nucleic acid (ANA), 2′-fluoro-ANA (FANA), or thiomorpholino.

In embodiments, at least 20, 30, 40, 50 60, 70 80 or 90% of the nucleotides in the ASO contain a modified sugar moiety.

In embodiments, the ASO comprises deoxyribonucleotides, ribonucleotides, or combinations thereof.

In embodiments, an ASO described herein comprises a gapmer structure or motif.

The present disclosure further provides a composition, such as a pharmaceutical composition, in an embodiment comprising and ASO described herein and a pharmaceutically acceptable carrier.

In further aspects, the present disclosure provides methods and uses of an ASO described herein for inhibiting SETBP1 expression in a cell, comprising contacting the ASO with the cell.

In embodiments, an ASO described herein is capable of inhibiting the expression of a mutant (i.e., disease-associated) allele, a healthy (i.e., non-disease-associated), allele, or both, of SETBP1.

In embodiments, the present disclosure provides methods and uses of an ASO described herein for preventing or treating a SETBP1-associated disease in a subject. In embodiments, the SETBP1-associated disease is Schinzel-Giedion Syndrome (SGS) or a SETBP1-associated cancer.

In embodiments, the methods and uses described herein may be performed in vitro, ex vivo, in vivo (in a subject), or a combination thereof.

### Definitions

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics, and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the specification unless otherwise indicated. See, e.g.: Sambrook J. and Russell D. Molecular Cloning: A Laboratory Manual, 3^{rd} ed., Cold Spring Harbor Laboratory Press, Cold Spring Harbor, N.Y. (2000); Ausubel F. M. et al., Short Protocols in Molecular Biology: A Compendium of Methods from Current Protocols in Molecular Biology, Wiley, John & Sons, Inc. (2002); Harlow E. and D. Lane, Using Antibodies: A Laboratory Manual; Cold Spring Harbor Laboratory Press, Cold Spring Harbor, N.Y. (1998); and Coligan J. E. et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The use of the terms “a” and “an” and “the” and similar referents in the context of describing the technology (especially in the context of the claims) are to be construed to cover both the singular and the plural, unless otherwise indicated herein or clearly contradicted by context.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein. For example, for the range of 18-20, the numbers 18, 19, and 20 are explicitly contemplated, and for the range 6.0-7.0, the numbers 6.0, 6.1, 6.2, 6.3, 6.4, 6.5, 6.6, 6.7, 6.8, 6.9, and 7.0 are explicitly contemplated. The terms “such as” are used herein to mean, and is used interchangeably with, the phrase “such as but not limited to”.

The use of any and all examples, or exemplary language (“e.g.”, “such as”) provided herein, is intended merely to better illustrate the technology and does not pose a limitation on the scope of the claimed invention unless otherwise claimed.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide”, and “protein” are used interchangeably to refer to a polymer of amino acid residues. The term also applies to amino acid polymers in which one or more amino acids are chemical analogues or modified derivatives of corresponding naturally-occurring amino acids.

“Coding sequence” or “encoding nucleic acid” as used herein means the nucleic acids (RNA or DNA molecule) that comprise a nucleotide sequence which encodes a protein or an ASO. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary. “Complementary” when expressed in terms of a minimal percentage means that at least that percentage of the nucleobases of the ASO and the nucleobases of a target sequence are capable of hydrogen bonding with one another when the nucleobase sequence of the ASO and the target sequence are aligned in opposing directions. Complementary nucleobases means nucleobases that are capable of forming hydrogen bonds with one another. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

“Gapmer” refers to a modified oligonucleotide comprising two external regions or “wings” and a central or internal region or “gap,” wherein the nucleosides comprising the internal gap region are chemically distinct from the nucleoside or nucleosides comprising the flanking wing regions. The three regions of a gapmer motif include the “5′ wing”, the “gap” and the “3 wing” which form a contiguous sequence of nucleosides wherein, in embodiments, at least some of the sugar moieties of the nucleosides of each of the wings differ from at least some of the sugar moieties of the nucleosides of the gap. Typically, at least the sugar moieties of the nucleosides of each wing that are closest to the gap (the 3-most nucleoside of the 5-wing and the 5-most nucleoside of the 3-wing) differ from the sugar moiety of the neighboring gap nucleosides, thus defining the boundary between the wings and the gap. In embodiments, the sugar moieties within the gap are the same as one another. In embodiments, the gap includes one or more nucleosides having a sugar moiety that differs from the sugar moiety of one or more other nucleosides of the gap. In embodiments, the sugar motifs of the two wings are the same as one another; in certain embodiments, the sugar motif of the 5-wing differs from the sugar motif of the 3-wing. in embodiments, the wings of a gapmer comprise 1-10, in further embodiments 5-10 nucleosides. In embodiments, the gap of a gapmer comprises 5-15 nucleosides, in further embodiments 5-10 nucleosides. In embodiments, the nucleosides on the gap side of each wing/gap junction are unmodified 2-deoxy nucleosides and the nucleosides on the wing sides of each wing/gap junction are modified nucleosides. Various gapmer configurations may be used. For example, the “gap” region may comprise modified internucleoside linkages (e.g., phosphorothioate) while the “wing” regions may comprise non-modified (i.e., phosphodiester) internucleoside linkages, or vice versa. In another example, the “gap” region may comprise deoxyribonucleotides which the “wing” regions may comprise ribonucleotides, further comprising a modified sugar moiety.

“Inhibit” as used herein refers to the ability to substantially antagonize, prohibit, prevent, suppress, decrease, slow, eliminate, stop, or reverse the progression or severity of the activity the targeted agent (e.g., expression of a target gene or sequence) or associated disease.

“Internucleoside linkage” is the covalent linkage between adjacent nucleosides in an oligonucleotide. As used herein, a “modified internucleoside linkage” means any internucleoside linkage other than a phosphodiester internucleoside linkage. A phosphorothioate linkage is a modified internucleoside linkage in which one of the nonbridging oxygen atoms of a phosphodiester internucleoside linkage is replaced with a sulfur atom.

### Sequence Similarity

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “substantially homologous” to another sequence if the two sequences are substantially identical and the functional activity of the sequences is conserved (as used herein, the term “homologous” does not infer evolutionary relatedness, but rather refers to substantial sequence identity, and thus is interchangeable with the terms “identity”/“identical”). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90%, or 95%. For the sake of brevity, the units (e.g., 66, 67, . . . 81, 82, . . . 91, 92%, . . . ) have not systematically been recited but are considered, nevertheless, within the scope of the present invention.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 96%, at least 98%, or at least 99%. Optimal alignment of sequences for comparisons of identity may be conducted using a variety of algorithms, such as the local homology algorithm of Smith and Waterman (Adv. Appl. Math. 2: 482-489, 1981), the homology alignment algorithm of Needleman and Wunsch (J. Mol. Biol. 48: 443-453, 1970), the search for similarity method of Pearson and Lipman (Proc. Natl. Acad. Sci. USA 85: 2444-2448, 1988), and the computerized implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described by Altschul et al. (J. Mol. Biol. 215: 403-410, 1990 using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably, stringent conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (Ausubel, F. M. et al., Current Protocols in Molecular Biology, John Wiley & Sons, New York, 2010). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (Ausubel, 2010, supra). Hybridization conditions may be modified in accordance with known methods depending on the sequence of interest (Tijssen P, Hybridization with nucleic acid probes, Part II, Volume 24, 1st Edition, Part II. Probe labeling and hybridization techniques, Elsevier Science, 1993, 344 pages). Generally, stringent conditions are selected to be about 5° C. lower than the thermal melting point for the specific sequence at a defined ionic strength and pH.

“Binding” refers to a sequence-specific, non-covalent interaction between macromolecules (e.g., between an ASO and its target sequence). Not all components of a binding interaction need be sequence-specific (e.g., contacts with phosphate residues in a DNA backbone), as long as the interaction as a whole is sequence-specific. “Affinity” refers to the strength of binding: increased binding affinity being correlated with a lower Kd.

“Recombination” refers to a process of the exchange of genetic information between two polynucleotides.

As used herein, a “target sequence” corresponds to a region of a polynucleotide within a cell that is capable of binding/hybridizing with an ASO described herein. A “target sequence” is within a “target gene” (e.g., SETBP1 or a fragment thereof) that is targeted by an ASO described herein. It corresponds to an endogenous gene naturally present within a cell. The target gene may comprise one or more mutations associated with a risk of developing a disease or disorder which may be corrected by for example inhibition of the expression of its corresponding polypeptide. One or both allele(s) of a target gene may be targeted within a cell, in accordance with the present disclosure.

A “target polynucleotide” as used herein refers to any endogenous polynucleotide or nucleic acid present in the genome of a cell and encoding or not a known gene product. “Target gene” as used herein refers to any endogenous polynucleotide or nucleic acid present in the genome of a cell and encoding a known or putative gene product. The target gene or target polynucleotide further corresponds to the polynucleotide within a cell whose expression can be inhibited by an ASO described herein. The target gene or target polynucleotide may be a mutated gene involved in a genetic disease.

“Promoter” as used herein means a synthetic or naturally-derived nucleic acid molecule which is capable of conferring, modulating, or controlling (e.g., activating, enhancing, and/or repressing) expression of a nucleic acid in a cell. A promoter may comprise one or more specific transcriptional regulatory sequences to further enhance or repress expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, CMV IE promoter, U6 promoter, a liver-specific promoter (e.g., LP1b; combining the human apolipoprotein E/C-I gene locus control region [ApoE-HCR] and a modified human α1 antitrypsin promoter [hAAT] coupled to an SV40 intron), human thyroxine binding globulin (TBG) promoter, CMV promoter, CAG promoter, CBH promoter, UbiC promoter, Ef1a promoter, H1 promoter, and 7SK promoter.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. In embodiments, an ASO described herein may be expressed from a transgene. Such a transgene may be administered to a subject in a DNA expression construct that is engineered to express an ASO in a subject. Such a DNA expression construct may be administered directly or using e.g., a viral vector.

In embodiments, a vector may comprise “regulatory” or “control” sequences, which refer to nucleic acid sequences necessary for the transcription and possibly translation of an operably linked coding sequence in a particular host cell. In addition to control sequences that govern transcription and translation, expression vectors may contain nucleic acid sequences that serve other functions as well.

In an embodiment, the vector further comprises a nucleic acid encoding a selectable marker or reporter protein. A selectable marker or reporter is defined herein to refer to a nucleic acid encoding a polypeptide that, when expressed, confers an identifiable characteristic (e.g., a detectable signal, resistance to a selective agent) to the cell permitting easy identification, isolation and/or selection of cells containing the selectable marker from cells without the selectable marker or reporter. Any selectable marker or reporter known to those of ordinary skill in the art is contemplated for inclusion as a selectable marker in the vector of the present disclosure. For example, the selectable marker may be a drug selection marker, an enzyme, or an immunologic marker. Examples of selectable markers or reporters include, but are not limited to, polypeptides conferring drug resistance (e.g., kanamycin/geneticin resistance), enzymes such as alkaline phosphatase and thymidine kinase, bioluminescent and fluorescent proteins such as luciferase, green fluorescent protein (GFP), yellow fluorescent protein (YFP), cyan fluorescent protein (CFP), blue fluorescent protein (BFP), citrine and red fluorescent protein from Discosoma sp. (dsRED), membrane bound proteins to which high affinity antibodies or ligands directed thereto exist or can be produced by conventional means, and fusion proteins comprising a membrane-bound protein appropriately fused to an antigen tag domain from, among others, hemagglutinin (HA) or Myc. The nucleic acid encoding the selectable marker or reporter protein may be under the control of the same promoter/enhancer as the nucleic acid of interest, or may be under the control of a distinct promoter/enhancer.

In embodiments, the vector may comprise additional elements, such as one or more origins of replication sites (often termed “ori”), restriction endonuclease recognition sites (multiple cloning sites, MCS), and/or internal ribosome entry site (IRES) elements.

Nucleic acids encoding ASOs of the present disclosure may be delivered into cells using one or more various vectors such as viral vectors. Accordingly, preferably, the above-mentioned vector is a viral vector for introducing an ASO of the present disclosure in a target cell. Non-limiting examples of viral vectors include retrovirus, lentivirus, herpesvirus, adenovirus, or adeno-associated virus (AAV), as well known in the art.

“Adeno-associated virus” or “AAV” as used interchangeably herein refers to a small virus belonging to the genus Dependoparvovirus of the Parvoviridae family that infects humans and some other primate species. AAV is not currently known to cause disease and, consequently, the virus may cause a very mild immune response.

In embodiments, the AAV vector preferably targets one or more cell types. Accordingly, the AAV vector may have enhanced cardiac, skeletal muscle, neuronal, liver, and/or pancreatic tissue (Langerhans cells) tropism. The AAV vector may be capable of delivering and expressing an ASO of the present disclosure in the cell of a mammal. For example, the AAV vector may be an AAV-SASTG vector (Piacentino et al., Hum. Gene Ther. 23: 635-646, 2012). The AAV vector may deliver ASOs to neurons, skeletal and cardiac muscle, and/or pancreas (Langerhans cells) in vivo. The AAV vector may be based on one or more of several capsid types, including AAV1, AAV2, AAV5, AAV6, AAV8, and AAV9. The AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery. In an embodiment, the AAV vector is a AAV-DJ vector. In an embodiment, the AAV vector is a AAV-DJ8 vector. In an embodiment, the AAV vector is a AAV2-DJ8 vector. In an embodiment, the AAV vector is a AAV-PHP.B vector. In an embodiment, the AAV vector is a AAV-PHP.B, AAV-9, or AAV-DJ8 (PHP.B: Deverman D. E. et al., Nat. Biotechnol. 34: 204-209, 2016; Jackson K. L. et al., Front. Mol. Neurosci. 9: 116, 2016; AAV DJ-8; and AAV9: Saraiva J. et al., J. Control. Release 241: 94-109, 2016; Inagaki K. et al., Mol. Ther. 14: 45-53, 2006).

A nucleic acid, expression cassette, or vector/plasmid described herein may be introduced into the cell using standard techniques for introducing nucleic acids into a cell, e.g., transfection, transduction, or transformation. In an embodiment, the vector is a viral vector, and the cell is transduced with the vector. As used herein, the term “transduction” refers to the stable transfer of genetic material from a viral particle (e.g., lentiviral) to a cell genome (e.g., hematopoietic cell genome). It also encompasses the introduction of non-integrating viral vectors into cells, which leads to the transient or episomal expression of the gene of interest present in the viral vector.

### Compositions

In another aspect, the present disclosure provides a composition (e.g., a pharmaceutical composition) comprising an ASO described herein. In an embodiment, the composition further comprises one or more biologically or pharmaceutically acceptable carriers, excipients, and/or diluents.

As used herein, “pharmaceutically acceptable” (or “biologically acceptable”) carriers, excipients, and/or diluents includes any and all solvents, dispersion media, coatings, antibacterial and antifungal agents, isotonic and absorption delaying agents, and the like that are physiologically compatible, and which can be used pharmaceutically or in biological systems. Such materials are characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

When the excipient serves as a diluent, it can be a solid, semisolid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders (see Remington: The Science and Practice of Pharmacy by Alfonso R. Gennaro, 2003, 21^{th} edition, Mack Publishing Company). In embodiments, the carrier may be suitable for intra-neural, parenteral, intravenous, intraperitoneal, intramuscular, subcutaneous, sublingual, or oral administration.

Some examples of suitable excipients include lactose, dextrose, sucrose, sorbitol, mannitol, starches, lecithin, phosphatidylcholine, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propyl-hydroxybenzoates; sweetening agents; and flavoring agents. The compositions of the disclosure can be formulated to provide quick sustained or delayed release of the active ingredient after administration to the patient by employing procedures known in the art.

Pharmaceutical compositions suitable for use in the disclosure include compositions wherein the active ingredients (e.g., an ASO described herein) are contained in an effective amount to achieve the intended purpose (e.g., preventing, treating, ameliorating, and/or inhibiting a disease or condition). The determination of an effective dose is well within the capability of those skilled in the art. For any compounds, the therapeutically effective dose can be estimated initially either in cell culture assays (e.g., cell lines) or in animal models, usually mice, rabbits, dogs, or pigs. The animal model may also be used to determine the appropriate concentration range and route of administration. Such information can then be used to determine useful doses and routes for administration in humans. An effective dose or amount refers to that amount of one or more active ingredient(s), which is sufficient for treating a specific disease or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time, and frequency of administration, drug combination(s), reaction sensitivities, and tolerance/response to therapy. Guidance as to particular dosages and methods of delivery is provided in the literature and generally available to practitioners in the art. In embodiments, dosages of an active ingredient of between about 0.01 and about 100 mg/kg body weight (in an embodiment, per day) may be used. In further embodiments, dosages of between about 0.5 and about 75 mg/kg body weight may be used. In further embodiments, dosages of between about 1 and about 50 mg/kg body weight may be used. In further embodiments, dosages of between about 10 and about 50 mg/kg body weight in further embodiments about 10, about 25 or about 50 mg/kg body weight, may be used.

In embodiments, an active ingredient (e.g., an ASO) described herein can be administered in various ways depending upon whether local or systemic treatment is desired and upon the area to be treated. Administration can be parenteral including intravenous, intraarterial, subcutaneous, intraperitoneal or intramuscular injection or infusion; or intracranial, e.g., intrathecal, intracerebroventricular, intraventricular, or intranasal administration. In another embodiment, the antisense or inhibitory nucleic acid is administered intrathecally or intra-cerebroventricular as a bolus injection. In an embodiment, an active ingredient (e.g., an ASO) described herein is administered or is for administration such that it comes into contact with a tissue or cell of the nervous system, in a further embodiment a CNS tissue or a CNS neuron. As used herein, the “central nervous system” or CNS is the portion of the nervous system comprising the brain and the spinal cord. By contrast, the “peripheral nervous system” or PNS is the portion of the nervous system other than the brain and the spinal cord. In an embodiment, the CNS tissue is the cerebral cortex, in a further embodiment, the hippocampus. As such, in embodiments an active ingredient (e.g., an ASO) described herein can be administered to treat CNS cells in vivo via direct intracranial or intrathecal injection or injection into the cerebrospinal fluid.

The present disclosure further provides a kit or package comprising at least one container means having disposed therein at least one of an ASO or composition as described herein. In an embodiment, the kit or package further comprises with instructions for use, such as for inhibition of SETBP1 expression in a cell, or for the prevention or treatment of a disease, disorder, or condition described herein, such as a SETBP1-associated disease.

### Methods/Uses

The present disclosure also relates to a method for inhibiting the expression of a nucleic acid of interest or gene of interest by a cell, the method comprising introducing an ASO, expression cassette, or vector described herein in the cell. In an embodiment, the cell is a primary cell, for example a brain/neuronal cell, a peripheral blood cell (e.g., a B or T lymphocyte, a monocyte, or a NK cell), a cord blood cell, a bone marrow cell, a cardiac cell, an endothelial cell, an epidermal cell, an epithelial cell, a fibroblast, hepatic cell, or a lung/pulmonary cell. In an embodiment, the cell is a bone marrow cell, peripheral blood cell, or cord blood cell.

In an embodiment, the gene of interest encodes a protein that is defective or absent in the cell.

The present disclosure also relates to a method for treating a disease, condition, or disorder in a subject, the method comprising administering a cell comprising a nucleic acid, expression cassette, or vector described herein. The present disclosure also relates to the use of a cell comprising a nucleic acid, expression cassette, or vector described herein method for treating a disease, condition, or disorder in a subject. The present disclosure also relates to the use of and ASO, expression cassette, or vector described herein method for the manufacture of a medicament for treating a disease, condition, or disorder in a subject. In an embodiment, the disease, condition, or disorder is associated with the expression of a defective (e.g., mutated) protein.

The disease or condition that is treated can be any in which expression of SETBP1 is associated with and/or involved in the etiology of the disease condition or disorder, e.g., causes, exacerbates or otherwise is involved in such disease, condition, or disorder. In embodiments, such diseases are referred to herein as a SETBP1-associated disease.

In embodiments, the SETBP1-associated disease is a SETBP1-associated neural (e.g., neurodevelopmental) disease, such as Schinzel-Giedion Syndrome (SGS). In embodiments, the SETBP1-associated disease is a disease or condition associated with malignancy or transformation of cells, e.g., cancer, such as an SGS tumour (e.g., sacral neuroepithelial tumour), or a hematological cancer, such as acute myeloid leukemia (AML), chronic myelomonocytic leukemia (CMML), myelodysplastic syndrome (MDS), solid tumors (e.g., bladder cancer), non-small cell lung cancer, gastric cancer, or melanoma.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, alleviation of symptoms, diminishment of any direct or indirect pathological consequences of the disease, preventing metastasis, decreasing the rate of or reversing disease progression, amelioration or palliation of the disease state, and remission or improved prognosis. The terms do not imply complete curing of a disease or complete elimination of any symptom or effect(s) on all symptoms or outcomes. In embodiments, an ASO describe herein may be used for preventing the occurrence or recurrence of disease, or delaying onset of disease.

In some embodiments, an ASO described herein is used as part of a combination treatment, such as with another therapy such as other chemotherapy, immunotherapy, radiotherapy, or surgery, according to the disease to be treated.

A “therapeutically effective amount” refers to an amount effective, at dosages and for periods of time necessary, to achieve the desired therapeutic result. A “prophylactically effective amount” refers to an amount effective, at dosages and for periods of time necessary, to achieve the desired prophylactic result, such as preventing or inhibiting the rate of onset or progression of the above-noted conditions. A prophylactically effective amount can be determined as described above for the therapeutically effective amount.

As used herein, the terms “subject” or “patient” are used interchangeably and are used to mean any animal, such as a mammal, including humans and non-human primates. In an embodiment, the above-mentioned subject is a mammal. In a further embodiment, the above-mentioned subject is a human.

### Antisense Oligonucleotides (ASOs)

In embodiments, ASOs described herein comprise one or more type of modified sugar and/or unmodified sugar moieties. In embodiments, modified sugar include for example replacement with a hexose ring (HNA), or a bicyclic ring, e.g., having a biradicle bridge between the C2 and C4 carbons on the ribose ring (LNA), or an unlinked ribose ring which typically lacks a bond between the C2 and C3 carbons (e.g. ULNA). Modified nucleosides also include nucleosides where e.g., the sugar moiety is replaced with a non-sugar moiety, for example in the case of peptide nucleic acids (PNA), or morpholino nucleic acids.

Modified sugars also include those in which the substituent groups on the ribose ring are altered to groups other than hydrogen, or the 2′-OH group naturally found in DNA and RNA nucleosides. Such substitution may for example be at the 2′, 3, 4′ or 5′ positions, Examples of 2′ substituted modified nucleosides are 2′-O-alkyl (e.g., 2′-O-methyl2′-O-alkyl-O-alkyl (e.g., 2′-methoxyethyl (2′-MOE)), 2′-fluoro, 2′-amino, and 2′-fluoro-ANA (FANA).

Certain modified sugar moieties comprise a substituent that bridges two atoms of the ribose ring to form a second ring, resulting in a bicyclic sugar moiety. For example, an LNA nucleoside comprises a bridge between the 2′ and 4′ sugar ring atoms which restricts the conformation of the ribose ring.

Modified sugars may also comprise different sugar rings. For example, morpholino oligonucleotides have nucleobases attached to six membered morpholine rings rather ribose.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include for example phosphates, which contain a phosphodiester bond (also referred to as unmodified or naturally occurring linkages), phosphotriesters, alkylphosphonates (e.g., methylphosphonates), phosphoramidates, phosphorothioates, and phosphorodithioates. Representative non-phosphorus containing internucleoside linking groups include for example methylenemethylimino, thiodiester, thionocarbamate, siloxane, and N,N-dimethylhydrazine.

In embodiments, modified oligonucleotides comprise one or more nucleosides comprising an unmodified nucleobase. In embodiments, modified oligonucleotides comprise one or more nucleoside comprising a modified nucleobase, which differs from a naturally occurring nucleobase. Naturally occurring nucleobases include for example adenine, guanine, cytosine, thymidine, uracil, xanthine and hypoxanthine. Modified nucleobases may for example contain a modified purine or pyrimidine, such as substituted purine or substituted pyrimidine (e.g., 5-methyl cytosine). In embodiments, a modified ASO described herein comprises one or more modified nucleobases.

In embodiments, an ASO described herein may be chemically linked or conjugated to one or more ligands or moieties that for example enhance the activity, targeting, cellular distribution and/or cellular uptake of the ASO.

In embodiments, the disclosure provides ASOs (modified or unmodified) that can be used to inhibit SETBP1 expression. In embodiments, such ASOs are one or more ASOs set forth in Table 1, which also shows corresponding SETBP1 target sequences.

TABLE 1
Nucleotide and target sequences of ASO's described herein

| | Description | | Description | | |
|---|---|---|---|---|---|
| | | SEQ | | SEQ | |
| ASO | Sequence (5′-3′) | ID NO: | Target seq in SETBP1 (5′-3′) | ID NO: | Exon |
| ASO1 | TGAGCCTAGTTCATCCTCCT | 27 | AGGAGGATGAACTAGGCTCA | 7 | 2 |
| ASO2 | GTTTGTGTTTCCTCTTTCCG | 28 | CGGAAAGAGGAAACACAAAC | 8 | 6 |
| ASO3 | TGCGGTTTGTGTTTCCTCTT | 29 | AAGAGGAAACACAAACCGCA | 9 | 6 |
| ASO4 | CCAAGAGCAGATCTTTGTTA | 30 | TAACAAAGATCTGCTCTTGG | 10 | 4 |
| ASO5 | TTTCTTACCCTGGGCCTTTG | 31 | CAAAGGCCCAGGGTAAGAAA | 11 | 4 |
| ASO6 | CTTTTCCACCACCGCTTTAA | 32 | TTAAAGCGGTGGTGGAAAAG | 12 | 4 |
| ASO7 | CAGAGTTGGAAAGTATCCTG | 33 | CAGGATACTTTCCAACTCTG | 13 | 4 |
| ASO8 | AAGGTTTCCCTGGACTCCAT | 34 | ATGGAGTCCAGGGAAACCTT | 14 | 2 |
| ASO3.1 | GCCTGCGGTTTGTGTTTCCTCTTTC | 35 | GAAAGAGGAAACACAAACCGCAGGC | 15 | 6 |
| ASO3.2 | GCGGGGGCCTGCGGTTTGTG | 36 | CACAAACCGCAGGCCCCCGC | 16 | 6 |
| ASO3.3 | TTGTGTTTCCTCTTTCCGCCT | 37 | AGGCGGAAAGAGGAAACACAA | 17 | 6 |
| ASO7.1 | CCCTCAGAGTTGGAAAGTAT | 38 | ATACTTTCCAACTCTGAGGG | 18 | 4 |
| ASO7.2 | GTTGG/AAAGTATCCTGTTAA | 39 | TTAACAGGATACTTTCCAAC | 19 | 4 |

### EXAMPLES

The present disclosure is illustrated in further detail by the following non-limiting examples.

### Example 1: Materials and Methods

### Somatic Cell Reprogramming

SGS and control cell lines were obtained through informed consent for participating in this research study through protocols approved by the institutional review board of the Douglas Hospital Research Center. Reprogramming of iPSCs and induction into NPCs followed previously described protocols generated in our lab [13]. Renal epithelial (RE) cells or peripheral blood mononuclear cells (PBMC) were reprogrammed by electroporation with episomal reprogramming vectors containing Oct4, Sox2, Myc3/4, Klf4, and ShRNA P53 (ALSTEM), and a puromycin resistance gene using a Neon Transfection System (Invitrogen, Burlington). Electroporation was conducted using 2-3×10⁵ cells and 3 μg of episomal vectors for each reaction. After electroporation, RE cells were plated on to Geltrex-coated tissue culture dish in REGM medium (Lonza). PBMCs were grown in StemSpan SFEM II (STEMCELL Technologies). After reprogramming, PBMCs were switched to ReproTesR medium (STEMCELL Technologies) until iPSCs formed and then changed to StemFlex media (Gibco). After 24 hours, the media was replaced with fresh medium containing 1 μg/mL puromycin (Sigma-Aldrich). After 48 hours, the media was replaced with TesR-E7 medium (STEMCELL Technologies), with daily media change. Observable iPSC colonies formed after 2 weeks in culture with TesR-E7 media. Colonies with a desirable size (500-1000 m in diameter) were then manually picked and replated into a new Geltrex-coated dish and cultured with TesR-E8 or mTeSR Plus medium (STEMCELL Technologies). Schinzel-Giedion Syndrome Tumour cells were obtained from the COMBINEDBrain Biorepository and grown in DMEM+15% FBS+1% NEAA. G870S SETBP1 overexpressing HEK293 FLP-In cell lines were obtained from our collaborators Piazza et al. and further described in [1] and grown in DMEM+10% FBS. All media were supplemented with 1% Antibiotic-Antimycotic (Gibco).

Quality Control for iPSCs

All iPSCs were rigorously assessed for contamination, pluripotency and genomic integrity using several assays. All cells were tested for mycoplasma contamination (EZ-PCR Mycoplasma Test Kit [Biological Industries]). Pluripotency was assessed by immunostaining with surface and nuclear pluripotency markers as previously described [14].

### Cortical Neural Progenitor Cell Induction

iPSCs were plated in Geltrex-coated tissue culture dishes at low density. When 15%-25% confluency was reached, iPSC culture media was replaced with Neural Induction Medium 1 (NIM1), composed of DMEM/F12 media supplemented with N2 (Invitrogen), B27 (Invitrogen), BSA (Gibco), SB431542 (Sten Cell Technologies), Noggin (GenScript) and Laminin (Sigma-Aldrich) with daily media change. On day 7, the medium was switched to Neural Induction Medium 2 (NIM2), composed of DMEM/F12 media supplemented with N2 (Invitrogen) B27 (Invitrogen), BSA (Gibco), NEAA (Gibco) and Laminin (Sigma-Aldrich) with daily media change. On day 12, cells were dissociated with Gentle Dissociation Reagent (Stem Cell Technologies) and suspended for 2-3 days for Neural Progenitor Cell (NPC) purification with STEMDiff Neural progenitor medium (STEMCELL Technologies) on non-adherent plates. Formed aggregates were then plated onto Geltrex-coated tissue culture dishes for NPC expansion and maintenance. Every 2-3 passages of NPCs, purification to remove non-NPC cells was performed. Cells were assessed for NPC morphology and stained for the presence of forebrain NPC markers as previously described [14].

### Cycloheximide Treatment

NPC cells were plated in Geltrex-coated 6 well plates at a density of 500,000 cells/well and allowed to recover overnight. 24 hours later, medium was switched to NPC medium containing 10 μM of cycloheximide (CHX) (Cayman Chemicals). Cells were harvested in RIPA buffer at the respective time points.

### Antisense Oligonucleotide Transfection

Antisense oligonucleotides (ASOs) with a phosphorothioate backbone modification were designed to specifically target different regions of the SETBP1 pre-mRNA sequence and were synthesized by Integrated DNA Technologies. ASO Control and ASOscr were used as negative controls. 6-FAM-ASO3 was used as a transfection control to ensure that ASOs transfected into the cells. 18-24 hours before transfection NPC cells were plated at 500,000 cells/well into 6-well plates. The following day, ASOs were transfected using TranslT-X2 (Mirus Bio) as per manufacturer's instructions. Forty-eight hours after transfection, cells were harvested and mRNA levels and protein levels were measured by quantitative RT-qPCR and western blotting respectively as described below.

### Plasmids and Transfection

SETBP1 was tagged with an HA epitope at the N-terminus and miniGFP at the C-terminus, connected by a short linker. The construct was cloned into a TET-inducible expression vector containing an hPGK promoter driving tdTomato expression. This construct is abbreviated as (HA-SETBP1). The constructs were transfected into HEK293 cells, and SETBP1 expression was induced with 1 μg/mL doxycycline for 24 hours.

### Quantitative Polymerase Chain Reaction

RNA samples are extracted using GENEzol TriRNA Pure Kit (Geneaid). Reverse transcriptions were done on the total RNA fraction, where 500 ng-1 ug of RNA was reverse transcribed into cDNA using the ProtoScript First Strand cDNA Synthesis Kit (NEB) according to the manufacturer's protocol. Real-time PCR amplifications were performed using the Luna Universal qPOR Master Mix (NEB) and gene specific primers in 384 well plates using the QuantStudio 7 Flex Real-Time PCR System (Applied Biosystems). For qPCR amplification, the following primers were used: SETBP1fwd (5′-GTCCACCTGAGATCAAGATC-3′; SEQ ID NO: 23), SETBP1 rev (5′-GTCCATGTGGTTCTGGCTGC-3′; SEQ ID NO: 24), GAPDH fwd (5′-GCTCTCTGCTCCTCCTGTTC-3′; SEQ ID NO: 25) and GAPDH rev (5′-CGACCAAATCCGTTGACTCC-3′; SEQ ID NO: 26). Relative quantification of mRNA was performed according to the comparative cycle threshold (CT) method using GAPDH expression as the endogenous control for normalization. Samples were assayed in triplicates.

### RNA Sequencing

Cells were lysed and RNA was extracted using the Genezol TriRNA Pure Kit (Geneaid). Total RNA was quantified using a NanoDrop Spectrophotometer ND-1000 (NanoDrop Technologies, Inc.) and its integrity was assessed through Agilent's TapeStation System using the 2100 Bioanalyzer (Agilent Technologies). RNA samples with RNA Integrity Number (RIN) values >9 were sequenced. PolyA enrichment library preparation and sequencing using NovaSeq 600 (100 bp paired-end reads) with 2500 million reads was performed. The RNA sequencing reads were processed using the nf-core RNAseq pipeline (version 3.14.0). The sequences from the SGS NPCs were aligned to the Homo sapiens GRCh38 reference genome, while the marmoset cell lines were aligned to the Callithrix jacchus genome assembly. Both alignments utilized the Ensembl release-111 and the corresponding GTF file of the same version. Subsequently, the resulting datasets were filtered to exclude genes with a mean count of fewer than 10 reads. Normalization and differential expression analysis were performed using the python implementation of the DESeq2 workflow (PyDESeq2). PyDESeq2, like DESeq2, normalizes RNAseq data by estimating size factors for each sample to adjust for differences in sequencing depth and RNA composition. After normalization, PyDESeq2 fits a negative binomial generalized linear model (GLM) to the normalized counts. This model accounts for biological and technical variability, ensuring that these factors do not impact the statistical significance of differential expression results. After fitting the model to the normalized count data, the Wald test is applied to each gene to test the null hypothesis that the coefficient (representing the effect of a condition or treatment) is equal to zero. This test provides a p-value for each gene, indicating whether there is a statistically significant difference in expression levels between conditions. The resulting p-values from the Wald test are then corrected for multiple testing using the Benjamini-Hochberg procedure, which controls the false discovery rate. This correction ensures that the list of differentially expressed genes is reliable and minimizes the risk of false positives. For the RNAseq analysis, the treatment of ASO3-MOE versus the scrambled version ASO3scr-MOE was of main interest.

### Immunoblotting

Cells were lysed using RIPA buffer supplemented with the cOmplete Protease Inhibitor Cocktail (Sigma-Aldrich). Protein concentrations were determined using Pierce BCA protein assay kit (ThermoFisher). 20 g of protein was loaded per well in Mini-PROTEAN TGX stain free precast gels (Bio-Rad) and run at 30 mA per gel for 45 minutes. Proteins were transferred to a nitrocellulose membrane using Trans-Blot Turbo Transfer system (Bio-Rad). The membranes were blocked in 5% non-fat milk in TBST buffer for 1 hour, followed by incubation with antibodies diluted in blocking solution overnight with shaking at 4° C. The following primary antibodies were used: SETBP1 (Proteintech #16841-1-AP) and Actin (Santa Cruz Biotechnology #sc-1616). Blots were washed thrice with TBST before they were incubated with respective secondary antibodies diluted in 5% non-fat milk in TBST for an hour at room temperature. Blots were washed thrice followed by band visualization using Clarity western ECL blotting substrates (Bio-Rad). Blots were imaged and analyzed using ImageLab software. The intensity of target bands was normalized to β-Actin.

### Quantification and Statistical Analysis

All experiments were performed in triplicate. The values are reported as mean fold induction and error bars on all graphs represent the mean±standard error of the mean (SEM). Significantly different values were determined using unpaired Student's t test when comparing two groups and One-way analysis of variance (ANOVA) followed by Tukey's post hoc test or Dunnett's post hoc test when comparing multiple groups. A probability value (p-value) of 0.05 or less was considered statistically significant. The asterisk (*) indicates whether there is a significant statistical increase or decrease compared to the mean of a specific condition: (*) p<0.05, (**) p<0.01, ***p<0.001, ****p<0.0001.

### Intracerebral Injection of ASO

On neonatal day 1 (P1), pups were anesthetized placed on a stereotaxic instrument. ASOs were dissolved in sterile PBS at the appropriate concentration to inject either 50 μg or 100 μg and a volume no bigger than 2 μL was injected by Intracerebroventricular (ICV) injection into the middle point between the lambda and bregma, ˜1 to 1.5 mm lateral from the sagittal midline. Mice were allowed to recover and monitored closely in the days following injection.

TABLE 2
ASOs of the studies described herein

| ASO | | | SEQ |
|---|---|---|---|
| name | Nucleotide Sequence (5′-3′) | Modification Description | ID NO: |
| ASO1 | 5′-T*G*A*G*CCTAGTTCATCC*T*C*C*T-3′ | * indicates a | 40 |
| | | phosphorothioate bond | |
| ASO2 | 5′-G*T*T*T*GTGTTTCCTCTT*T*C*C*G-3′ | * indicates a | 41 |
| | | phosphorothioate bond | |
| ASO3 | 5′-T*G*C*G*GTTTGTGTTTCC*T*C*T*T-3′ | * indicates a | 42 |
| | | phosphorothioate bond | |
| ASO4 | 5′-C*C*A*A*GAGCAGATCTTT*G*T*T*A-3′ | * indicates a | 43 |
| | | phosphorothioate bond | |
| ASO5 | 5′-T*T*T*C*TTACCCTGGGCC*T*T*T*G-3′ | * indicates a | 44 |
| | | phosphorothioate bond | |
| ASO6 | 5′-C*T*T*T*TCCACCACCGCT*T*T*A*A-3′ | * indicates a | 45 |
| | | phosphorothioate bond | |
| ASO7 | 5′-C*A*G*A*GTTGGAAAGTAT*C*C*T*G-3′ | * indicates a | 46 |
| | | phosphorothioate bond | |
| ASO8 | 5′-A*A*G*G*TTTCCCTGGACT*C*C*A*T-3′ | * indicates a | 47 |
| | | phosphorothioate bond | |
| ASO | 5′-G*T*G*C*A*A*C*A*G*C*A*C*C*T*G*G*A*A*G*G-3′ | * indicates a | 48 |
| control | | phosphorothioate bond | |
| ASO3- | 5′-#T#G#C#G#G*T*T*T*G*T*G*T*T*T*C#C#T#C#T#T-3′ | * indicates a | 49 |
| LNA | | phosphorothioate bond | |
| | | # indicates a locked | |
| | | nucleic acid base | |
| ASO3sc | 5′-#T#C#T#C#T*G*C*T*G*T*G*G*T*T*G#T#T#C#T#T-3′ | * indicates a | 50 |
| r-LNA | | phosphorothioate bond | |
| | | # indicates a locked | |
| | | nucleic acid base | |
| ASO3- | 5′-6- | + indicates a 2′-O- | 51 |
| fluor | FAM+rT+rG+rC+rG+rGT*T*T*G*T*G*T*T*T*C*+rC+rT+rC+rT+rT-3′ | methoxy-ethyl Bases (2′- | |
| | | MOE) | |
| | | r indicates a RNA base | |
| | | * indicates a | |
| | | phosphorothioate bond | |
| ASO3.1 | 5′- | + indicates a 2′-O- | 52 |
| | +rG++C+rC+rT+rGC*GG*TT*T*G*T*G*TT*TCC*T+rC+rT+rT+rT+rC-3′ | methoxy-ethyl Bases (2′- | |
| | | MOE) | |
| | | r indicates a RNA base | |
| | | * indicates a | |
| | | phosphorothioate bond | |
| ASO3.2 | 5′- | + indicates a 2′-O- | 53 |
| | +rG+rC+rG+rG+rGG*G*C*C*T*G*C*G*G*T*+rT+rT+rG+rT+rG-3′ | methoxy-ethyl Bases (2′- | |
| | | MOE) | |
| | | r indicates a RNA base | |
| | | * indicates a | |
| | | phosphorothioate bond | |
| ASO3.3 | 5′- | + indicates a 2′-O- | 54 |
| | +T+rT+rG+rT+rGT*T*T*C*C*T ***** T*T*C*+C++G+C+C+rT-3′ | methoxy-ethyl Bases (2′- | |
| | | MOE) | |
| | | r indicates a RNA base | |
| | | * indicates a | |
| | | phosphorothioate bond | |
| ASO3- | 5′- | + indicates a 2′-O- | 55 |
| MOE | +rT+rG+rC+rG+rG/T*T*T*G*T*G*T*T*T*C*+rC+rT+rC+rT+rT-3′ | methoxy-ethyl Bases (2′- | |
| | | MOE) | |
| | | r indicates a RNA base | |
| | | * indicates a | |
| | | phosphorothioate bond | |
| ASO3sc | 5′- | + indicates a 2′-O- | 56 |
| r-MOE | +rT+rT+rT+rG+rTT*G*C*G*T*T*G*T*C*T*+rC+rT+rT+rG+rC-3′ | methoxy-ethyl Bases (2′- | |
| | | MOE) | |
| | | r indicates a RNA base | |
| | | * indicates a | |
| | | phosphorothioate bond | |
| ASO7.1 | 5′-+rC+rC+rC+rT+rCA*G*A*G*T*T*G*G*A*A*+rA+rG+rT+rA+rT-3′ | + indicates a 2′-O- | 57 |
| | | methoxy-ethyl Bases (2′- | |
| | | MOE) | |
| | | r indicates a RNA base | |
| | | * indicates a | |
| | | phosphorothioate bond | |
| ASO7.2 | 5′-+rG+T+T+rG+rG/A*A*A*G*T*A*T*C*C*T*++G+rT+rT+A+rA-3′ | + indicates a 2′-O- | 58 |
| | | methoxy-ethyl Bases (2′- | |
| | | MOE) | |
| | | r indicates a RNA base | |
| | | * indicates a | |
| | | phosphorothioate bond | |
| ASO7- | 5′-+rC++A+G+A+rGT*T*G*G*A*A*A*G*T*A*+rT+C+rC+rT+rG-3′ | + indicates a 2′-O- | 59 |
| MOE | | methoxy-ethyl Bases (2′- | |
| | | MOE) | |
| | | r indicates a RNA base | |
| | | * indicates a | |
| | | phosphorothioate bond | |
| ASO7sc | 5′-+rG+rC+rT+rA+rTA*T*T*A*G*G*C*G*A*T*+rA+rC+rG+rA+rG-3′ | + indicates a 2′-O- | 60 |
| r-MOE | | methoxy-ethyl Bases (2′- | |
| | | MOE) | |
| | | r indicates a RNA base | |
| | | * indicates a | |
| | | phosphorothioate bond | |

TABLE 3
Mutations and cell types

| Cell line name | Cell type | Mutation |
|---|---|---|
| Control A1 | NPC | WT |
| Control A2 | NPC | WT |
| Control A3 | NPC | WT |
| SGS1 | NPC | c.2612 T > G, p.I871S |
| SGS2 | NPC | c.2608 G > A, p.G870S |
| SGS3 | NPC | c.2601C > A, p.S867R |
| HEK293-G870S | HEK293 | c.2608 G > A, p.G870S |
| SGS tumour | Neuroepithelial tumour | c.2612 T > C, p. I871T |
| HEK293-HA-SETBP1 | HEK293 | Overexpressing SETBP1 |

### Example 2: SETBP1 Protein Half-Life is Increased in SGS NPCs Due to Reduced Protein Degradation

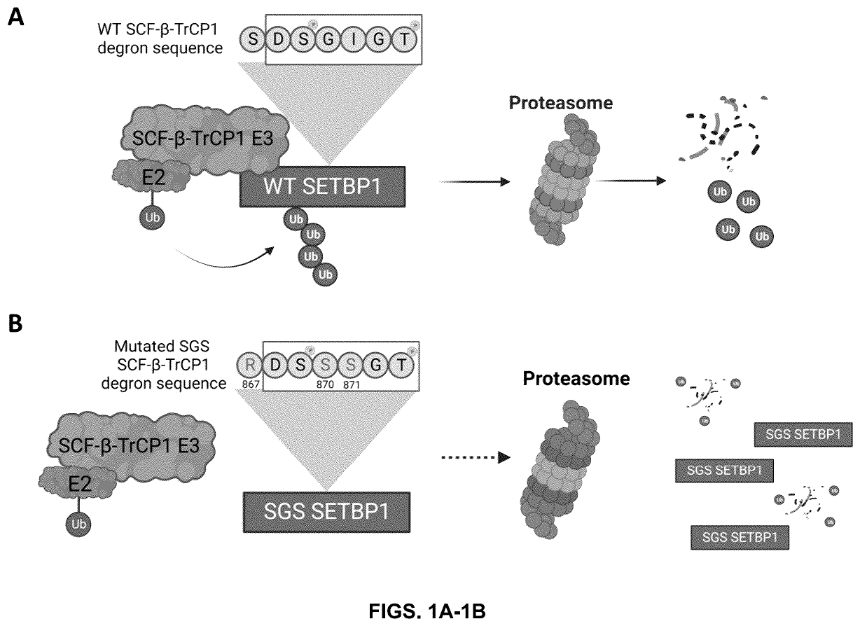

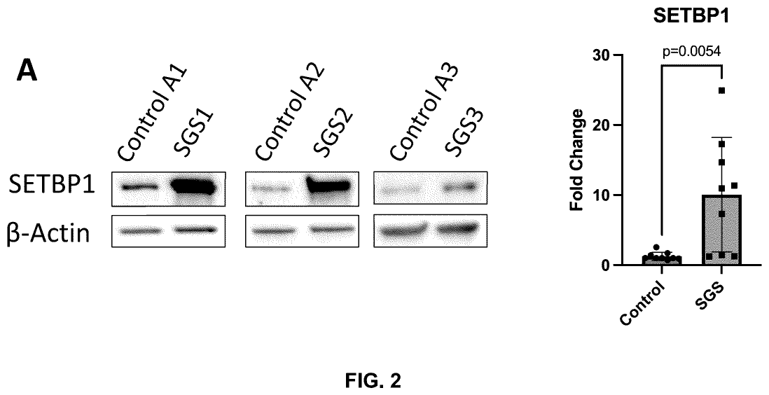

It has been reported [2, 15-17] that mutations within a mutational hotspot in the degron motif of SETBP1 confer the mutant protein resistance to degradation by the SCF-PTrCP1 E3 ligase, which is part of the ubiquitin-proteasome system, causing the protein to accumulate (FIG. 1A). SGS and sex-matched control NPCs were assayed to observe whether there was an increase in the SETBP1 amount of these cells Western blot quantification shows that there is on average; an 8-fold increase of SETBP1 in SGS cells as compared to their respective family controls (FIG. 2).

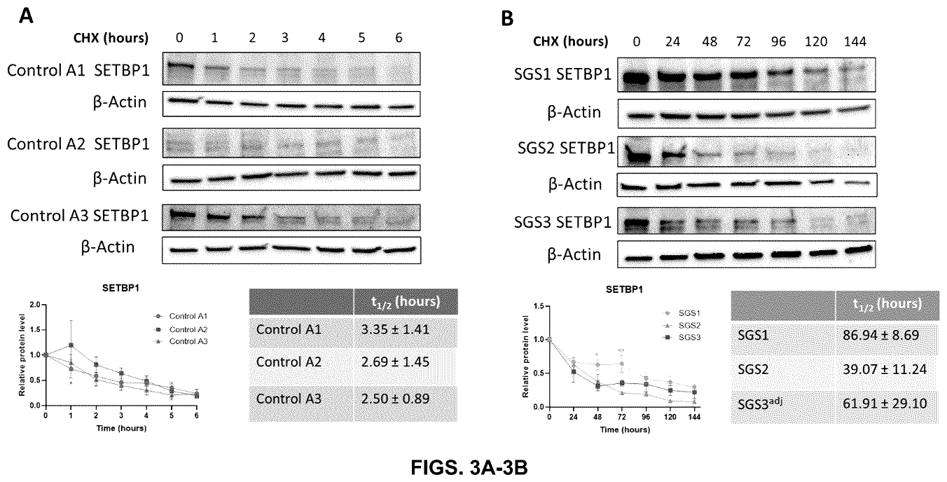

Given that the degron motif sequence is changed by SGS-causing mutations, we wanted to further understand whether there were stability and degradation rate differences between WT and 3GS mutant SETBP1. Control NPCs were treated with protein synthesis inhibitor cycloheximide (CHX) and the amount of time it took for SETBP1 protein expression to decrease was measured every hour. SETBP1 levels begin to decrease on average after 2.85±1.25 hours of protein synthesis inhibition in control cells while SGS SETBP1 protein levels remain stable after 6 hours of CHX treatment (FIG. 3A), Thus, SGS SETBP1 levels were measured following treatment for up to 144 hours, collecting cell lysates every 24 hours, SGS SETBP1 half-life was found to be on average around ˜62.64±49.03 hours, showing a broad variation in the half-life of SETBP1 according to the mutation present (FIG. 3B). These data demonstrate that mutant SETBP1 protein is more stable than WT SETBP1 and that its degradation happens primarily through the ubiquitin-proteasome degradation system, since degradation still occurs when the degron motif is mutated albeit less efficiently.

### Example 3: Identification of ASO3 and ASO7 as Candidates for SETBP1 Downregulation

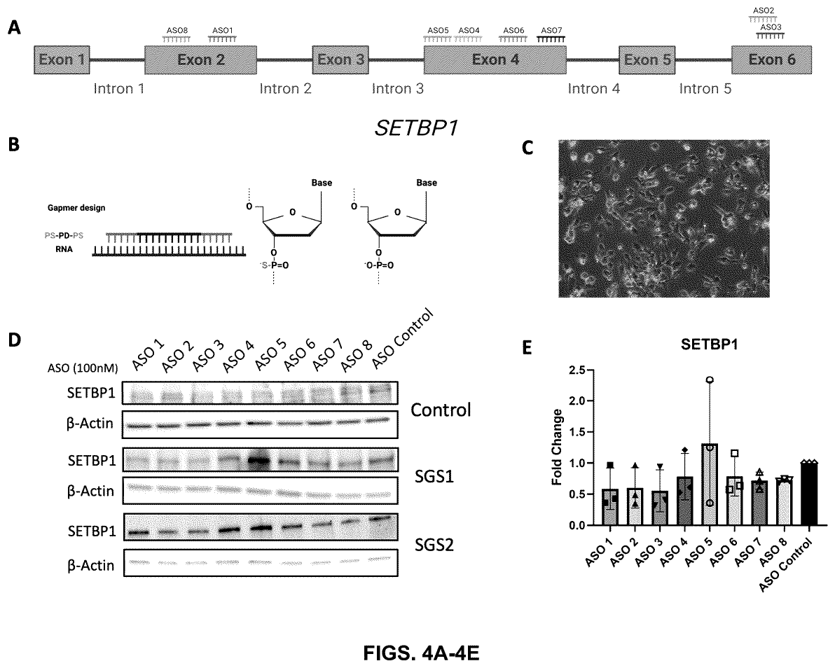

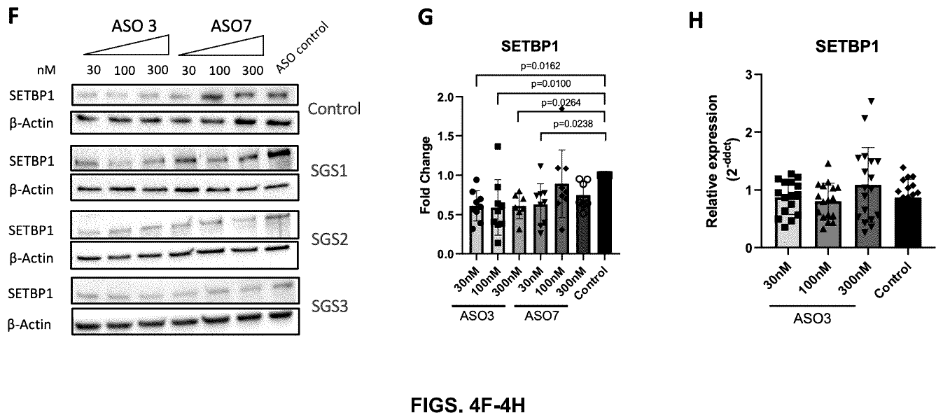

Based on the studies described herein, we propose ASO treatment as a therapy option for SGS patients to reduce their seizures, decrease their suffering, stop further brain atrophy, and decrease tumours or tumor size. ASOs developed and tested to reduce SETBP1 expression against SETBP1 may have the added benefit of potentially being able to treat myeloid leukemias caused by somatic mutations at the identical locus. We established iPSC-derived NPCs as an in vitro screening tool to analyze ASOs for their efficiency at reducing SETBP1 protein levels. We performed a screen of 8 different ASOs with a chimeric phosphorothioate/phosphodiester (PS/PD) backbone (FIGS. 4A and 4B) and tested them in 2 SGS and 1 control NPC lines (FIGS. 4D and 4E). SGS NPCs were transfected with individual ASOs at a concentration of 100 nM using TranslT-X2 transfection reagent. Transfection efficiency was assessed through the use of a 6-FAM tagged ASO (ASOfluor) (FIG. 4C). ASO3 and ASO7 were identified as candidates for further testing and were subsequently transfected into all 3 SGS NPC lines as well as a control NPC line at increasing doses for 2 days. WB of SETBP1 following transfection indicates that ASO3 and ASO7 successfully downregulate expression of SETBP1 at different doses albeit ASO3 does so more successfully (FIGS. 4F and 4G). Although the PT/PS gapmer design was selected with the goal of activating the RNAse H degradation pathway of the mRNA/ASO duplex, qPCR measurement of SETBP1 mRNA levels indicates that SETBP1 gene expression in not reduced in response to ASO treatment (FIG. 4H), pointing towards a steric blocking form of action.

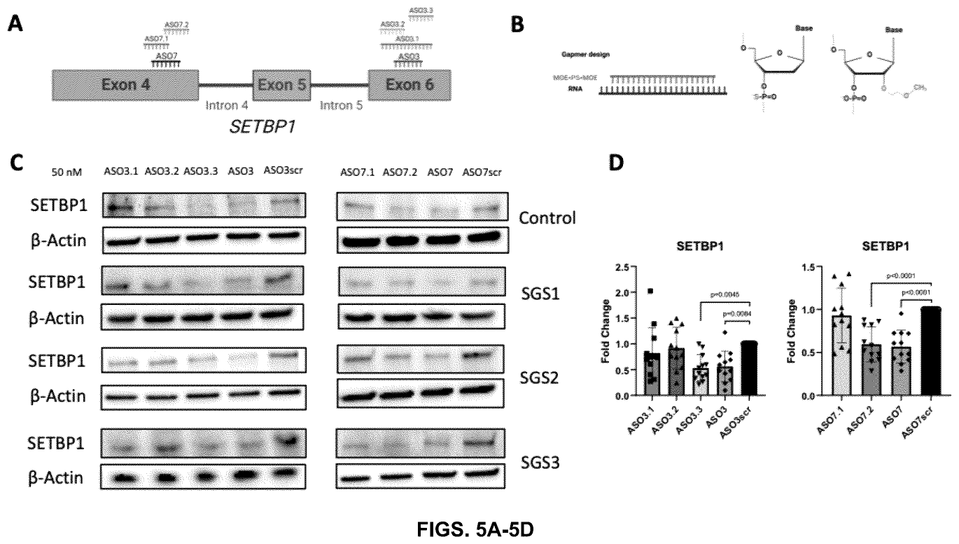

For the next stage of testing, we performed a modification to the backbone of ASO3 and ASO7 into a 2′-Methoxyethyl RNA (2′MOE)/phosphorothioate backbone (MOE/PS) designed to minimize immune reaction from the body, be more stable and increase potency (FIG. 5B). We also designed tiled ASOs around the regions targeted by ASO3 and ASO7 to identify additional ASO candidates that could downregulate SETBP1 and be tested further (FIG. 5A). WB assessment following transfection determined that ASO3.3 and ASO3 significantly downregulated SETBP1 protein expression in SGS and control NPCs (FIG. 5C) when compared to their respective scrambled control. Similarly, we found that ASO7.2 and ASO7 efficiency downregulated SETBP1 protein expression in SGS and control NPCs (FIG. 5D).

### Example 4: Candidate ASOs Downregulate SETBP1 Protein Expression in Various Disease Models In Vitro

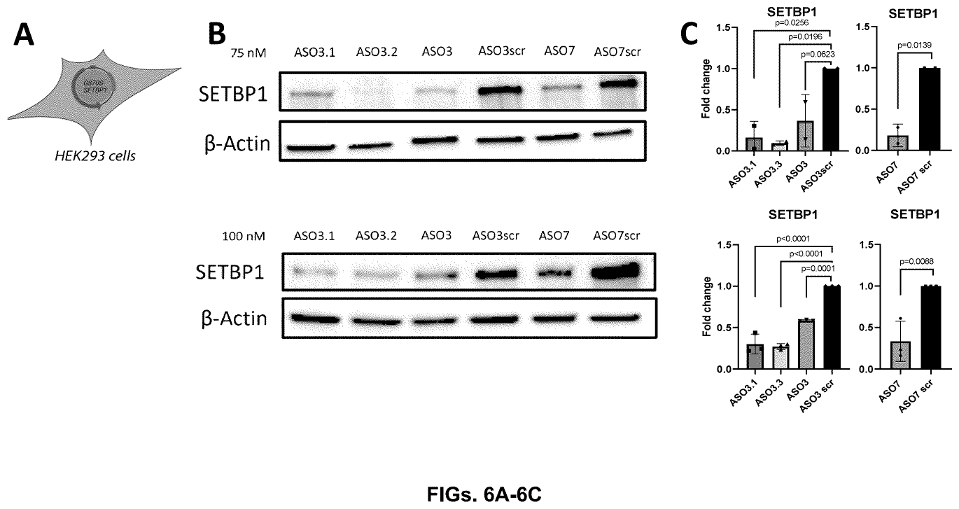

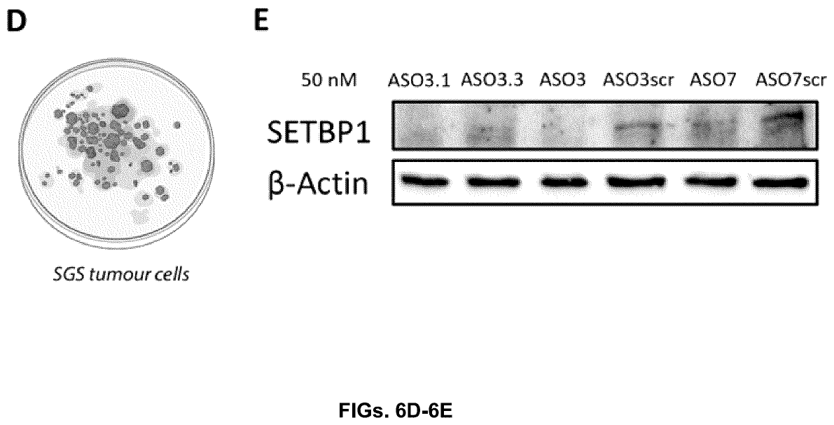

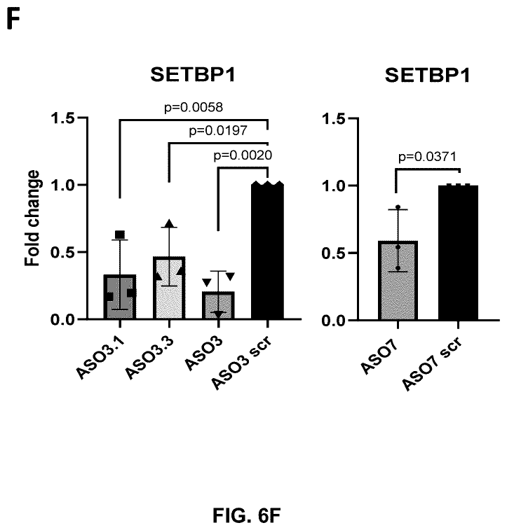

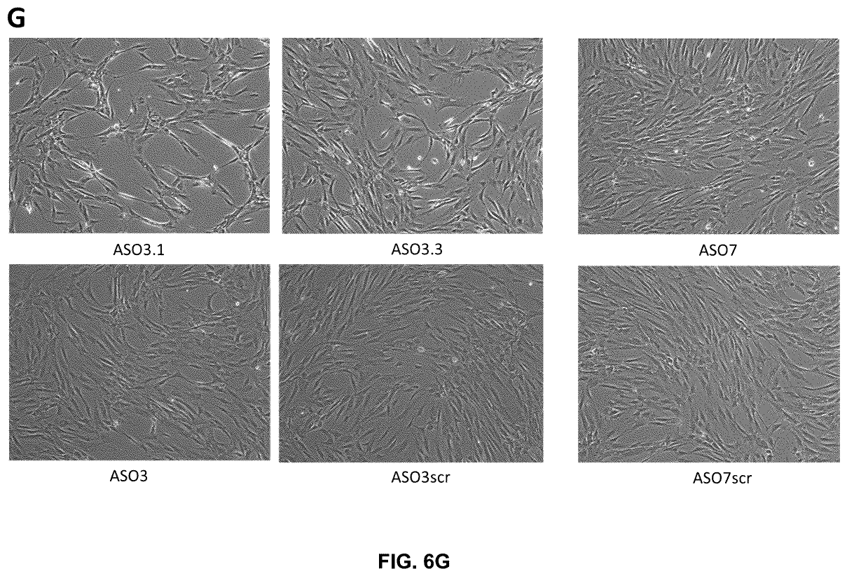

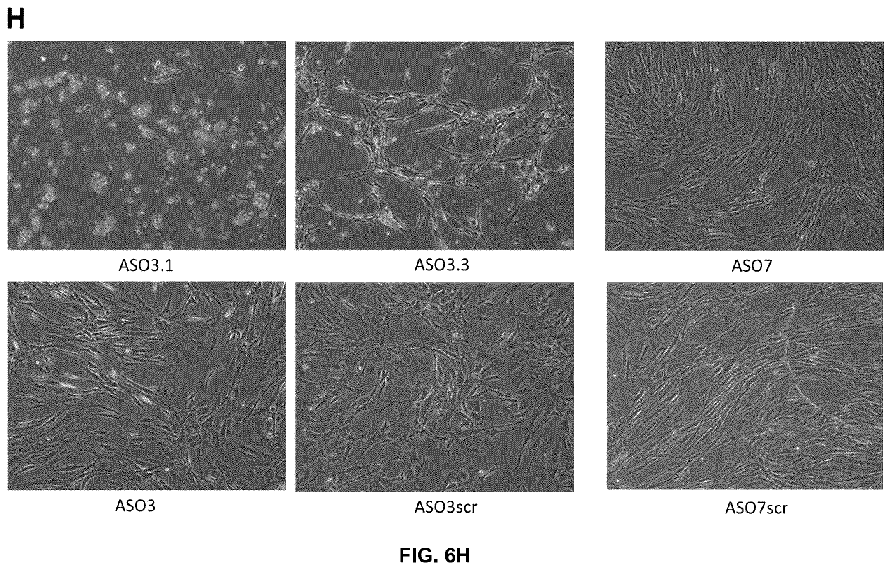

We further confirmed the efficiency of our ASO by testing their action on other models of disease. ASOs were transfected into a HEK293 FLP-In cell lines stably overexpressing the G870S mutation in SETBP1 (FIG. 6A). It can be seen that ASO3, ASO3.1, ASO3.3 and ASO7 at different doses can robustly decrease G870S-SETBP1 levels in these cells (FIGS. 6B and 6C). We obtained cells from a sacral neuroepithelial tumour (SGS tumour) removed from a child with SGS and cultured in vitro (FIG. 6D). ASOs were tested on this cell line and it was observed that ASO3.1, ASO3.3, ASO3 and ASO7 significantly downregulate SETBP1 in these cells (FIGS. 6E and 6F). Interestingly, ASO3.1 had a particularly strong toxicity effect on SGS tumour cells, where after 2 days of treatment at 50 nM, cells have a visible reduction in number (FIG. 6G) and after 4 days, most cells have been killed by treatment (FIG. 6H). A similar effect was not found when NPCs were treated with ASO3.1. This data suggests that ASO3.1 could be a good candidate to selectively treat SGS tumours and decrease their size.

### Example 5: ASO-induced SETBP1 Downregulation at the RNA Level

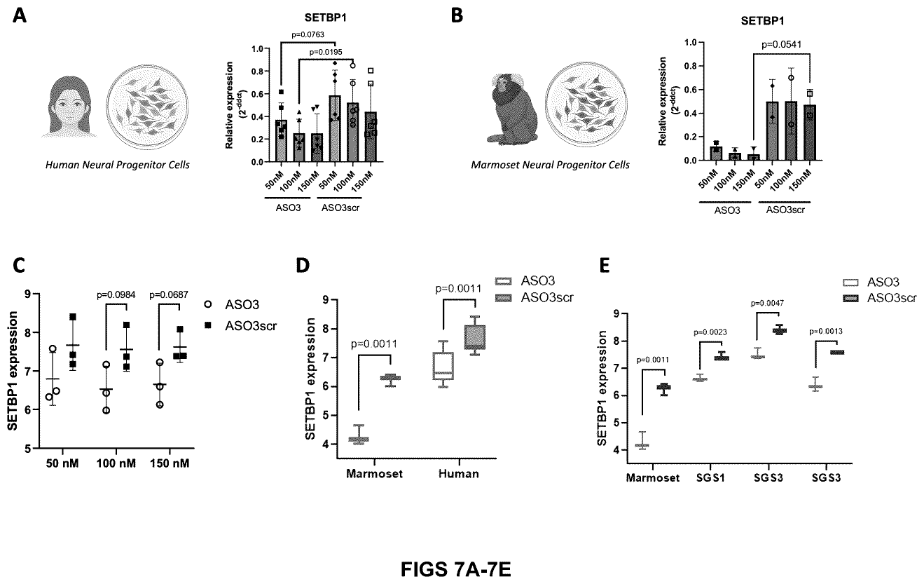

We next assessed ASO treatment at the RNA level and ASO3-MOE was chosen as the lead candidate. Three SGS NPC lines were transfected with increasing doses (50, 100 and 150 nM) of ASO3-MOE or ASO3scr-MOE. QPCR analysis confirmed that ASO3-downregulates SETBP1 mRNA in human SGS NPC lines (FIG. 7A). Since the marmoset (Callithrix jacchus) is a good model to test safety and knockdown efficiency of ASOs in a non-human primate species to support future in vivo testing for pre-clinical studies, we also treated healthy marmoset NPCs similarly with increasing doses of ASO3 (FIG. 7B). We next performed a transcriptomic analysis on NPCs from 3 SGS and 1 marmoset cell lines treated with increasing doses (50, 100 and 150 nM) of ASO3-MOE or ASO3scr-MOE. Individual doses showed reduction of SETBP1 when comparing ASO3-MOE and ASO3scr-MOE (FIG. 7C). Pooled dose expression data in SGS NPCs demonstrated that there was a significant overall reduction in SETBP1 expression following ASO3-MOE administration irrespective of dose used (FIG. 7D). Individual assessment of SETBP1 downregulation in each individual cell line demonstrates that there is variation in SETBP1 levels across individuals at baseline state, but the reduction of SETBP1 levels is significant in each line following ASO treatment (FIG. 7E). We conclude that ASO3-MOE significantly reduces SETBP1 mRNA levels in vitro in SGS patients and healthy marmosets.

### Example 6: Anti-SETBP1 ASO Pharmacokinetics

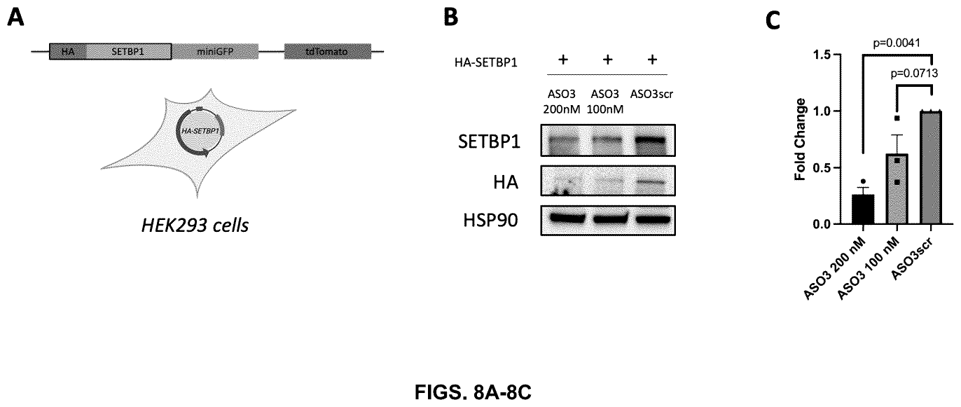

To understand how ASO3-MOE might act as a drug, we engineered an HA-tagged SETBP1 expression vector (HA-SETBP1), and asked whether ASO3 shows reduction of SETBP1 protein from a transfected overexpression plasmid (FIG. 8A). Transfection of both ASO and plasmid provides unambiguous evidence for ASO efficiency against SETBP1. We observed significant depletion of SETBP1 of ˜40% at 100 nM and 60% at 200 nM (FIGS. 8B, C).

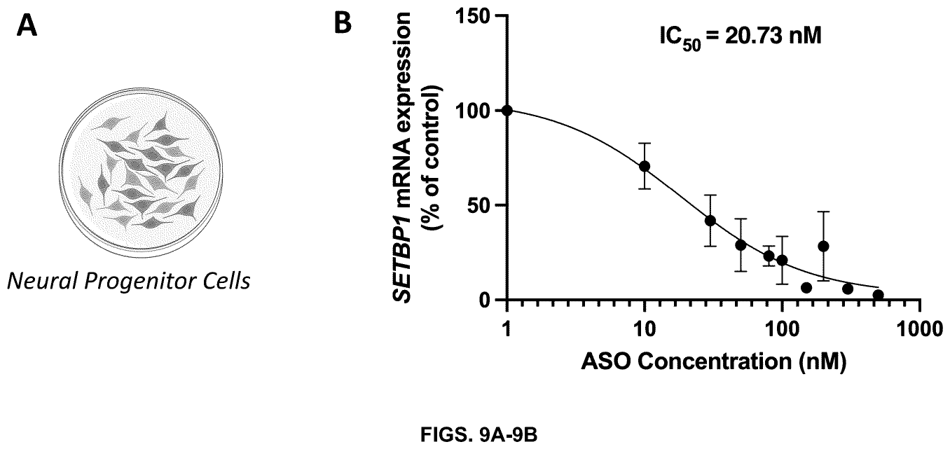

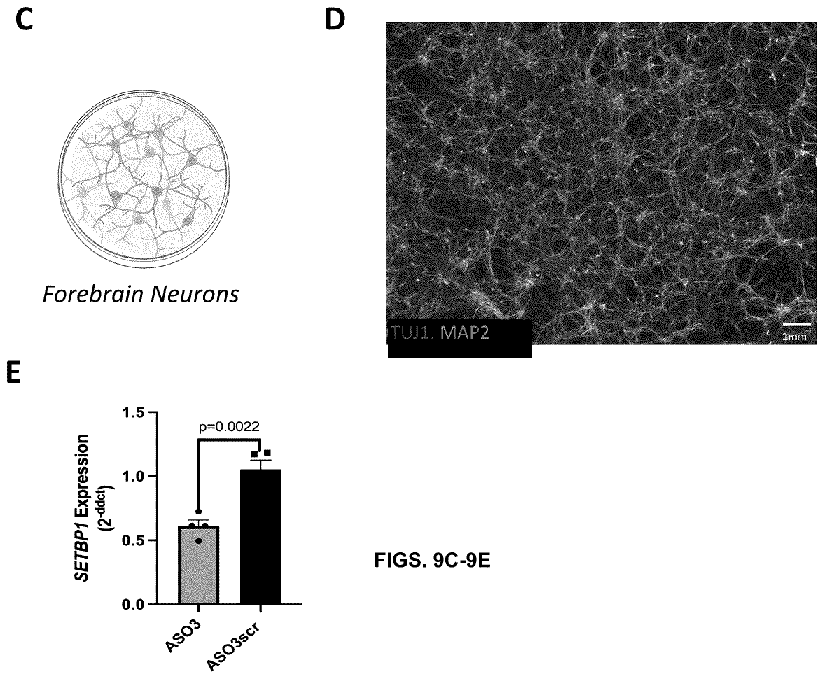

To test the effect of increasing doses of ASO3-MOE and to determine the IC50 of ASO3-MOE, we induced iPSCs to forebrain NPCs from 3 SGS cases (FIG. 9A) and assessed SETBP1 mRNA downregulation via qPCR at 10 doses in the nM range. Doses ranged from 10 nM to 500 nM and we performed each assessment in triplicate in each SGS patient line. From these experiments, we calculate an IC₅₀ value of 20.73 nM for ASO3-MOE in human forebrain NPCs (FIG. 9B). Doses up to 200 nM did not visibly induce cell death in NPCs, whereas doses of 300 nM and 500 nM resulted in moderate cell death (not shown), though this may be due to increased transfection reagent.

To demonstrate ASO3-MOE efficacy in a non-dividing cell, we transfected 3-week old forebrain neurons made using our published protocols [18] from 4 SGS patients with ASO3-MOE (FIG. 9C, D). Preliminary experiments in neurons from a single individual suggested a higher dose of ASO3 was required in neurons than in NPCs (data not shown). Using a dose of 300 nM, and 4 SGS patient cell lines in triplicate, we confirm a SETBP1 protein reduction of ˜45% after 48 hours of ASO3-MOE exposure in post-mitotic neurons from 4 different SGS patients in triplicate (FIG. 9E).

### Example 7: Candidate ASOs are Safe In Vivo and Distribute Throughout the Brain

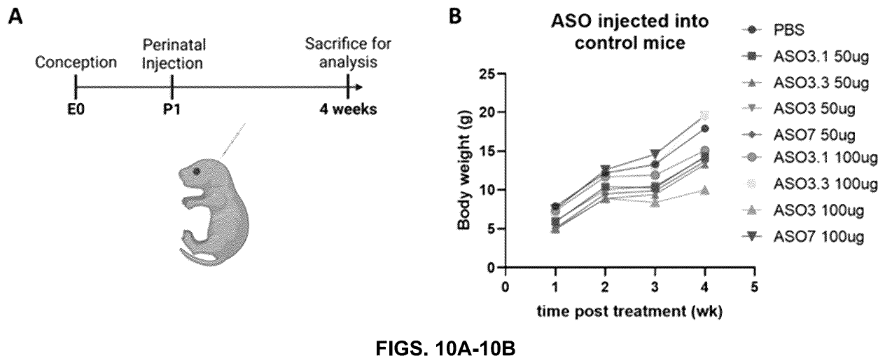

To assess the safety and survival of neonatal mice following ASO injection, we performed an in vivo go/no-go assessment of the selected ASOs in control mice (FIG. 10). One mouse per condition was injected with a single dose of 50 μg or 100 μg of each respective ASO through IntraceCerebroVentricular (ICV) injection at P1 and assessed for up to 4 weeks (FIG. 10A). Following 24 hrs after injection and beyond, mice didn't develop any seizures and survived past 4 weeks of age. We didn't observe any big fluctuations in mouse weight and differences in weight at 4 weeks of age among mice could be attributed to sex differences (FIG. 10B, Table 4).

TABLE 4
Weight, sex and experimental condition data
following ASO ICV injection into P1 mice

| | | | | | Body Weight (g) | | | |
|---|---|---|---|---|---|---|---|---|
| Mouse | Sex | Treatment | Dose | | 1 week | 2 week | 3 week | 4 week |
| 1 | M | Vehicle (PBS) | 2 | μl | 7.9 | 12.2 | 13.3 | 17.9 |
| 2 | F | ASO3 | 100 | μg | 5.1 | 8.9 | 8.4 | 10.0 |
| 3 | M | ASO3.3 | 100 | μg | 7.3 | 12.0 | 13.2 | 19.6 |
| 4 | M | ASO7 | 100 | μg | 7.5 | 12.6 | 14.6 | 19.6 |
| 5 | F | ASO3.1 | 100 | μg | 7.3 | 11.7 | 11.9 | 15.1 |
| 6 | F | ASO3 | 50 | μg | 6.0 | 10.0 | 10.5 | 14.2 |
| 7 | F | ASO3.3 | 50 | μg | 4.9 | 8.9 | 9.4 | 13.3 |
| 8 | F | ASO7 | 50 | μg | 5.1 | 9.5 | 9.9 | 13.7 |
| 9 | F | ASO3.1 | 50 | μg | 5.9 | 10.4 | 10.3 | 14.3 |

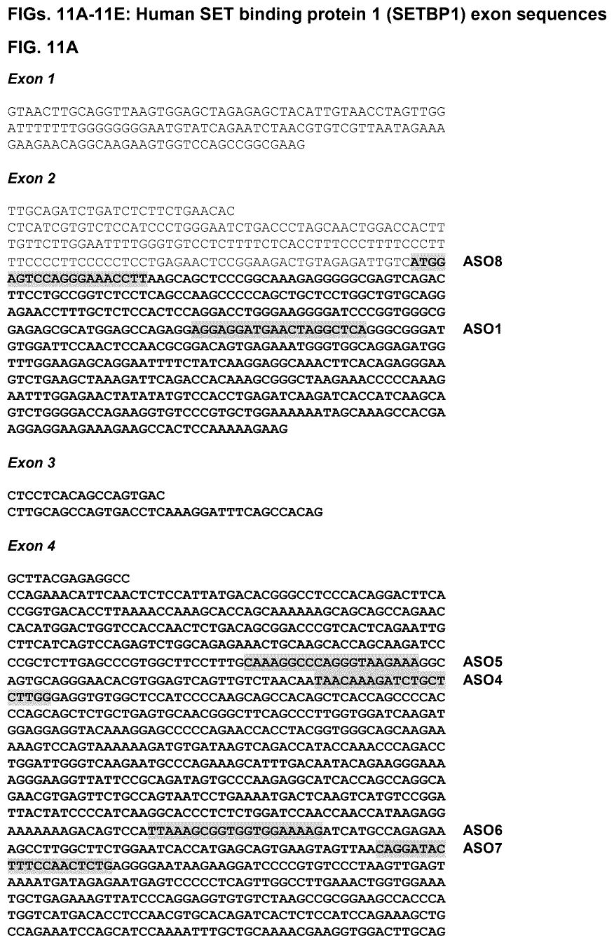

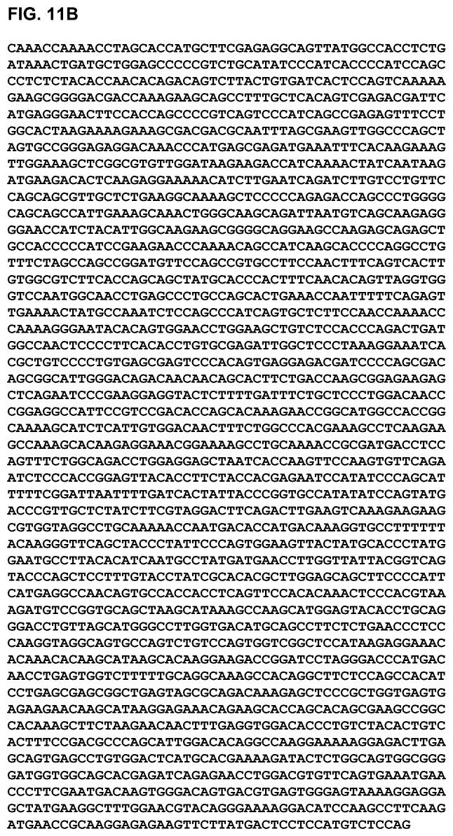

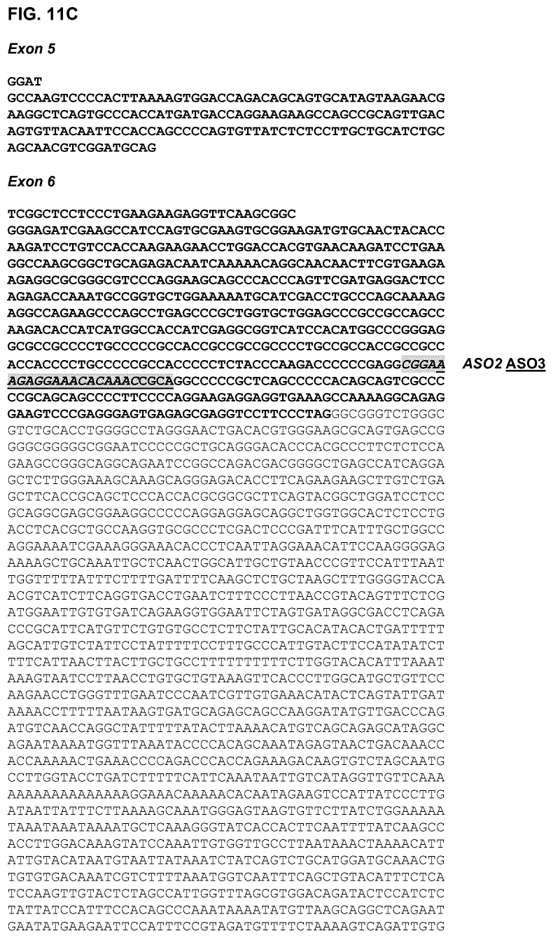

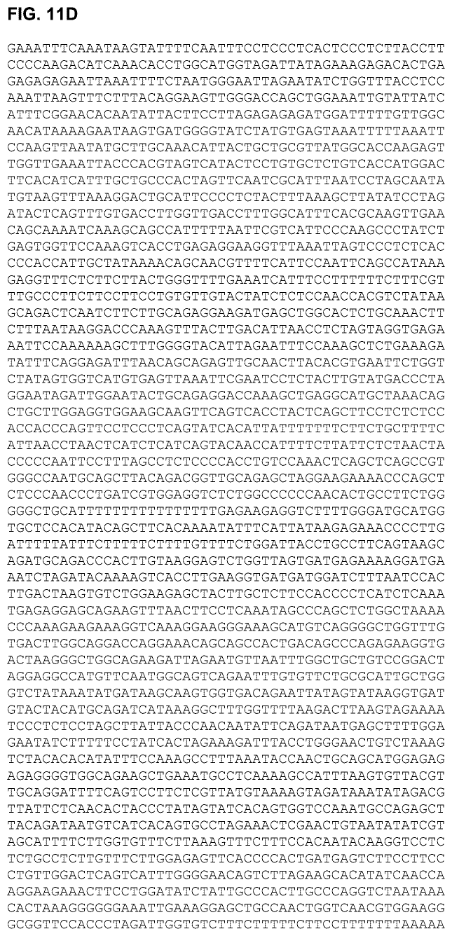

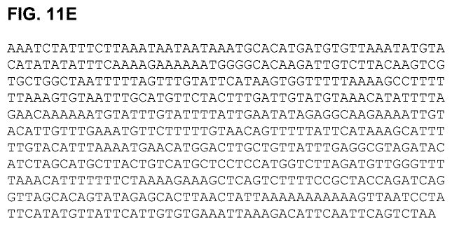

TABLE 5
Summary of sequences described herein

| SEQ ID | |
|---|---|
| NO(s): | Description |
| 1-6 | SETBP1 exons 1-6 (FIG. 11) |
| 7-14 | SETBP1 target sequences corresponding to ASO1, ASO2, ASO3, ASO4, ASO5, ASO6, ASO7, and |
| | ASO8 (FIG. 11) |
| 15-17 | SETBP1 target sequences corresponding to ASO3.1, ASO3.2 and ASO3.3 |
| 18-19 | SETBP1 target sequences corresponding to ASO7.1 and ASO7.2 |
| 20 | SETBP1 DNA sequence containing exons 1-6 |
| 21 | SETBP1 coding sequence (FIG. 12) |
| 22 | SETBP1 amino acid sequence (FIG. 13) |
| 23-24 | SETBP1 fwd and rev primers |
| 25-26 | GAPDH fwd and rev primers |
| 27-34 | Nucleotide sequences corresponding to ASO1, ASO2, ASO3, ASO4, ASO5, ASO6, ASO7, and ASO8 |
| | (Table 1) |
| 35-37 | Nucleotide sequences corresponding to ASO3.1, ASO3.2 and ASO3.3 (Table 1) |
| 38-39 | Nucleotide sequences corresponding to ASO7.1 and ASO7.2 (Table 1) |
| 40-47 | ASO1-ASO8 (Table 2) |
| 48 | ASO control (Table 2) |
| 49 | ASO3-LNA (Table 2) |
| 50 | ASO3scr-LNA (Table 2) |
| 51 | ASO-fluor (Table 2) |
| 52-55 | ASO3.1, ASO3.2, ASO3.3 and ASO3-MOE (Table 2) |
| 56 | ASO3scr-MOE (Table 2) |
| 57-59 | ASO7.1, ASO7.2 and ASO7-MOE (Table 2) |
| 60 | ASO7scr-MOE (Table 2) |
| 61-62 | Nucleotide and amino acid sequences defining the SKI-homology domain |
| 63 | Target sequence defined by positions 1-1366 of SEQ ID NO: 21 |
| 64 | Target sequence defined by positions 1-1370 of SEQ ID NO: 21 |
| 65 | Target sequence defined by positions 1-859 of SEQ ID NO: 21 |
| 66 | Target sequence defined by positions 782-1366 of SEQ ID NO: 21 |
| 67 | Target sequence defined by positions 782-1370 of SEQ ID NO: 21 |
| 68 | Target sequence defined by positions 1-195 of SEQ ID NO: 21 |
| 69 | Target sequence defined by positions 782-859 of SEQ ID NO: 21 |
| 70 | Target sequence defined by positions 1271-1366 of SEQ ID NO: 21 |
| 71 | Target sequence defined by positions 1271-1370 of SEQ ID NO: 21 |
| 72 | Target sequence defined by positions 1343-1370 of SEQ ID NO: 21 |
| 73 | Target sequence defined by positions 4647-4673 of SEQ ID NO: 21 |
| 74 | Target sequence defined by positions 4647-4682 of SEQ ID NO: 21 |
| 75 | WT SCF-β-TrCP1 degron sequence (FIG. 1) |
| 76 | Mutated SGS-β-TrCP1 degron sequence (FIG. 1) |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

ADDIN Mendeley Bibliography CSL_BIBLIOGRAPHY 1. Piazza, R., et al., SETBP1 induces transcription of a network of development genes by acting as an epigenetic hub. Nature communications, 2018. 9(1): p. 2192.
2. Piazza, R., et al., Recurrent SETBP1 mutations in atypical chronic myeloid leukemia. Nat Genet, 2013. 45(1): p. 18-24.
3. Nguyen, N., et al., Myb expression is critical for myeloid leukemia development induced by Setbp1 activation. Oncotarget, 2016. 7(52): p. 86300-86312.
4. Acuna-Hidalgo, R., et al., Overlapping SETBP1 gain-of-function mutations in Schinzel-Giedion syndrome and hematologic malignancies. PLoS genetics, 2017. 13(3): p. e1006683.
5. Meszaros, B., et al., Degrons in cancer. Sci Signal, 2017. 10(470).
6. McPherson, E., Schinzel-Giedion midface retraction syndrome. Atlas Genet Cytogenet Oncol Haematol., 2006. 10(4): p. 292-294.
7. Coccaro, N., et al., SETBP1 dysregulation in congenital disorders and myeloid neoplasms. Oncotarget, 2017. 8(31): p. 51920-51935.
8. Filges, I., et al., Reduced expression by SETBP1 haploinsufficiency causes developmental and expressive language delay indicating a phenotype distinct from Schinzel-Giedion syndrome. Journal of medical genetics, 2011. 48(2): p. 117-122.
9. Beschorner, R., et al., Extradural ependymal tumor with myxopapillary and ependymoblastic differentiation in a case of Schinzel-Giedion syndrome. Acta Neuropathol, 2007. 113(3): p. 339-46.
10. Wild, E. J. and S. J. Tabrizi, Therapies targeting DNA and RNA in Huntington's disease. The Lancet Neurology, 2017. 16(10): p. 837-847.
11. Roovers, J., P. De Jonghe, and S. Weckhuysen, The therapeutic potentialof RNA regulation in neurological disorders. Expert opinion on therapeutic targets, 2018. 22(12): 1017-1028.
12. Kohyanagi, N., et al., The impact of SETBP1 mutations in neurological diseases and cancer, Genes to Cells, 2023, 28: 629-641.
13. Bell, S., et al., A rapid pipeline to model rare neurodevelopmental disorders with simultaneous CRISPR/Cas9 gene editing. Stem cells translational medicine, 2017. 6(3): p. 886-896.
14. Hettige, N. C., et al., FOXG1 targets BMP repressors and cell cycle inhibitors in human neural progenitor cells. Human Molecular Genetics, 2023: p. ddad089.
15. Makishima, H., et al., Somatic SETBP1 mutations in myeloid malignancies. Nature Genetics, 2013. 45(8): p. 942.
16. Makishima, H., Somatic SETBP1 mutations in myeloid neoplasms. Int J Hematol, 2017. 105(6): p. 732-742.
17. Hoischen, A., et al., De novo mutations of SETBP1 cause Schinzel-Giedion syndrome. Nature Genetics, 2010. 42(6): p. 483.
18. Bell, S., et al., Differentiation of human induced pluripotent stem cells (iPSCs) into an effective model of forebrain neural progenitor cells and mature neurons. Bio-protocol, 2019. 9(5): p. e3188-e3188.

1. A modified antisense oligonucleotide (ASO) comprising a sequence that is at least 80% complementary to a target sequence within the SETBP1 nucleic acid sequence of SEQ ID NO: 20.

2. The modified ASO of claim 1, comprising a sequence that is 100% complementary to a target sequence within the SETBP1 nucleic acid sequence of SEQ ID NO: 20.

3. The modified ASO of claim 1, wherein the target sequence comprises at least 5 contiguous nucleotides of SEQ ID NO: 20.

4. The modified ASO of claim 1, which is 10-30 nucleotides in length.

5. The modified ASO of claim 1, wherein the target sequence is within a coding sequence of the SETBP1 nucleic acid sequence of SEQ ID NO: 20.

6. The modified ASO of claim 1, wherein the target sequence is within exon 2, 4 or 6 of the SETBP1 nucleic acid sequence of SEQ ID NO: 20.

7. The modified ASO of claim 1, wherein the target sequence is within a region of SEQ ID NO: 20, wherein the region is defined by a 5′ end up to 100 nucleotides 5′ from the nucleotide sequence of one or more of SEQ ID NOs: 7-19 and a 3′ end up to 100 nucleotides 3′ from the nucleotide sequence of one or more of SEQ ID NOs: 7-19 and 63-74.

8. The modified ASO of claim 1, wherein the target sequence is within a region defined by the nucleotide sequence of one or more of SEQ ID NOs: 63-74.

9. The modified ASO of claim 1, wherein the modified ASO comprises one or more modified internucleoside linkages, and/or one or more modified sugars, and/or one or more modified nucleobases.

10. The modified ASO of claim 9, wherein the one or more modified internucleoside linkages comprise one or more of phosphorothioate, phosphorodithioate, phosphoramidate, phosphonocarboxylate, and thiophosphonocarboxylate, alkylphosphonate.

11. The modified ASO of claim 9, wherein the one or more modified sugars comprise one or more sugar modifications comprising one or more of 2′-O-alkyl, 2′-deoxy, 2′-O-alkyl-O-alkyl, 2′-fluoro, 2′-amino, 2′-arabinosyl nucleotide, 2′-F-arabinosyl nucleotide, locked nucleic acid (LNA) nucleotide, 2′-amido bridge nucleic acid, unlocked nucleic acid (ULNA) nucleotide, 4′-thioribosyl nucleotide, constrained ethyl (cET), arabino nucleic acid (ANA), 2′-fluoro-ANA (FANA), and thiomorpholino.

12. The modified ASO of claim 1, comprising a gapmer structure comprising an internal gap region flanked by 5′ and 3′ wing regions, wherein the internal and wing regions differ at least by nucleoside type and/or internucleoside linkage type.

13. The modified ASO of claim 12, wherein the internal gap region comprises DNA nucleosides and the flanking wing regions comprise RNA nucleosides.

14. The modified ASO of claim 12, wherein each of the internal gap region and flanking wing regions comprises 5-10 nucleosides.

15. The modified ASO of claim 1, wherein the target sequence is set forth in Table 1, and/or wherein the ASO is set forth in Table 1 or 2.

16. A composition comprising the modified ASO of claim 1 and a pharmaceutically acceptable carrier.

17. A method of inhibiting SETBP1 expression in a cell, comprising contacting the cell with the modified ASO of claim 1.

18. A method for treating or preventing a disease in a subject, comprising administering a therapeutically or prophylactically effective amount of the modified ASO of claim 1 to a subject suffering from or susceptible to the disease.

19. The method of claim 18, wherein the disease is a SETBP1-associated disease.

20. The method of claim 18, wherein the disease is Schinzel-Giedion Syndrome or a SETBP1-associated cancer.


SEQUENCE LISTING (76 sequences)

SEQ ID NO: 1 - DNA, 134 bp, Homo sapiens
  source 1..134: mol_type = other DNA
gtaacttgcaggttaagtggagctagagagctacattgtaacctagttggatttttttggggggggaatgtatcagaatctaacgtgtcgttaatagaaagaagaacaggcaagaagtggtccagccggcgaag

SEQ ID NO: 2 - DNA, 658 bp, Homo sapiens
  source 1..658: mol_type = other DNA
ttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaag

SEQ ID NO: 3 - DNA, 54 bp, Homo sapiens
  source 1..54: mol_type = other DNA
ctcctcacagccagtgaccttgcagccagtgacctcaaaggatttcagccacag

SEQ ID NO: 4 - DNA, 3460 bp, Homo sapiens
  source 1..3460: mol_type = other DNA
gcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctgaggggaataagaaggatccccgtgtccctaagttgagtaaaatgatagagaatgagtccccctcagttggccttgaaactggtggaaatgctgagaaagttatcccaggaggtgtgtctaagccgcggaagccacccatggtcatgacacctccaacgtgcacagatcactctccatccagaaagctgccagaaatccagcatccaaaatttgctgcaaaacgaaggtggacttgcagcaaaccaaaacctagcaccatgcttcgagaggcagttatggccacctctgataaactgatgctggagcccccgtctgcatatcccatcaccccatccagccctctctacaccaacacagacagtcttactgtgatcactccagtcaaaaagaagcggggacgaccaaagaagcagcctttgctcacagtcgagacgattcatgagggaacttccaccagccccgtcagtcccatcagccgagagtttcctggcactaagaaaagaaagcgacgacgcaatttagcgaagttggcccagctagtgccgggagaggacaaacccatgagcgagatgaaatttcacaagaaagttggaaagctcggcgtgttggataagaagaccatcaaaactatcaataagatgaagacactcaagaggaaaaacatcttgaatcagatcttgtcctgttccagcagcgttgctctgaaggcaaaagctcccccagagaccagccctggggcagcagccattgaaagcaaactgggcaagcagattaatgtcagcaagaggggaaccatctacattggcaagaagcggggcaggaagccaagagcagagctgccacccccatccgaagaacccaaaacagccatcaagcaccccaggcctgtttctagccagccggatgttccagccgtgccttccaactttcagtcacttgtggcgtcttcaccagcagctatgcacccactttcaacacagttaggtgggtccaatggcaacctgagccctgccagcactgaaaccaatttttcagagttgaaaactatgccaaatctccagcccatcagtgctcttccaaccaaaacccaaaagggaatacacagtggaacctggaagctgtctccacccagactgatggccaactccccttcacacctgtgcgagattggctccctaaaggaaatcacgctgtcccctgtgagcgagtcccacagtgaggagacgatccccagcgacagcggcattgggacagacaacaacagcacttctgaccaagcggagaagagctcagaatcccgaaggaggtactcttttgatttctgctccctggacaacccggaggccattccgtccgacaccagcacaaagaaccggcatggccaccggcaaaagcatctcattgtggacaactttctggcccacgaaagcctcaagaagccaaagcacaagaggaaacggaaaagcctgcaaaaccgcgatgacctccagtttctggcagacctggaggagctaatcaccaagttccaagtgttcagaatctcccaccggagttacaccttctaccacgagaatccatatcccagcatttttcggattaattttgatcactattacccggtgccatatatccagtatgacccgttgctctatcttcgtaggacttcagacttgaagtcaaagaagaagcgtggtaggcctgcaaaaaccaatgacaccatgacaaaggtgccttttttacaagggttcagctaccctattcccagtggaagttactatgcaccctatggaatgccttacacatcaatgcctatgatgaaccttggttattacggtcagtacccagctcctttgtacctatcgcacacgcttggagcagcttccccattcatgaggccaacagtgccaccacctcagttccacacaaactcccacgtaaagatgtccggtgcagctaagcataaagccaagcatggagtacacctgcagggacctgttagcatgggccttggtgacatgcagccttctctgaaccctcccaaggtaggcagtgccagtctgtccagtggtcggctccataagaggaaacacaaacacaagcataagcacaaggaagaccggatcctagggacccatgacaacctgagtggtctttttgcaggcaaagccacaggcttctccagccacatcctgagcgagcggctgagtagcgcagacaaagagctcccgctggtgagtgagaagaacaagcataaggagaaacagaagcaccagcacagcgaagccggccacaaagcttctaagaacaactttgaggtggacaccctgtctacactgtcactttccgacgcccagcattggacacaggccaaggaaaaaggagacttgagcagtgagcctgtggactcatgcacgaaaagatactctggcagtggcggggatggtggcagcacgagatcagagaacctggacgtgttcagtgaaatgaacccttcgaatgacaagtgggacagtgacgtgagtgggagtaaaaggaggagctatgaaggctttggaacgtacagggaaaaggacatccaagccttcaagatgaaccgcaaggagagaagttcttatgactcctccatgtctccag

SEQ ID NO: 5 - DNA, 171 bp, Homo sapiens
  source 1..171: mol_type = other DNA
ggatgccaagtccccacttaaaagtggaccagacagcagtgcatagtaagaacgaaggctcagtgcccaccatgatgaccaggaagaagccagccgcagttgacagtgttacaattccaccagccccagtgttatctctccttgctgcatctgcagcaacgtcggatgcag

SEQ ID NO: 6 - DNA, 5432 bp, Homo sapiens
  source 1..5432: mol_type = other DNA
tcggctcctccctgaagaagaggttcaagcggcgggagatcgaagccatccagtgcgaagtgcggaagatgtgcaactacaccaagatcctgtccaccaagaagaacctggaccacgtgaacaagatcctgaaggccaagcggctgcagagacaatcaaaaacaggcaacaacttcgtgaagaagaggcgcgggcgtcccaggaagcagcccacccagttcgatgaggactccagagaccaaatgccggtgctggaaaaatgcatcgacctgcccagcaaaagaggccagaagcccagcctgagcccgctggtgctggagcccgccgccagccaagacaccatcatggccaccatcgaggcggtcatccacatggcccgggaggcgccgcccctgcccccgccaccgccgccgcccctgccgccaccgccgccaccacccctgcccccgccaccccctctacccaagaccccccgaggcggaaagaggaaacacaaaccgcaggcccccgctcagcccccacagcagtcgcccccgcagcagccccttccccaggaagaggaggtgaaagccaaaaggcagaggaagtcccgagggagtgagagcgaggtccttccctagggcgggtctgggcgtctgcacctggggcctagggaactgacacgtgggaagcgcagtgagccggggcgggggcggaatcccccgctgcagggacacccacgcccttctctccagaagccgggcaggcagaatccggccagacgacggggctgagccatcaggagctcttgggaaagcaaagcagggagacaccttcagaagaagcttgtctgagcttcaccgcagctcccaccacgcggcgcttcagtacggctggatcctccgcaggcgagcggaaggcccccaggaggagcaggctggtggcactctcctgacctcacgctgccaaggtgcgccctcgactcccgatttcatttgctggccaggaaaatcgaaagggaaacaccctcaattaggaaacattccaaggggagaaaagctgcaaattgctcaactggcattgctgtaacccgttccatttaattggtttttatttcttttgattttcaagctctgctaagctttggggtaccaacgtcatcttcaggtgacctgaatctttcccttaaccgtacagtttctcgatggaattgtgtgatcagaaggtggaattctagtgataggcgacctcagacccgcattcatgttctgtgtgcctcttctattgcacatacactgatttttagcattgtctattcctatttttcctttgcccattgtacttccatatatcttttcattaacttacttgctgccttttttttttcttggtacacatttaaataaagtaatccttaacctgtgctgtaaagttcacccttggcatgctgttccaagaacctgggtttgaatcccaatcgttgtgaaacatactcagtattgataaaacctttttaataagtgatgcagagcagccaaggatatgttgacccagatgtcaaccaggctatttttatacttaaaacatgtcagcagagcataggcagaataaaatggtttaaataccccacagcaaatagagtaactgacaaaccaccaaaaactgaaaccccagacccaccagaaagacaagtgtctagcaatgccttggtacctgatctttttcattcaaataattgtcataggttgttcaaaaaaaaaaaaaaaaaggaaacaaaaacacaatagaagtccattatcccttgataattatttcttaaaagcaaatgggagtaagtgttcttatctggaaaaataaataaataaaatgctcaaagggtatcaccacttcaattttatcaagccaccttggacaaagtatccaaattgtggttgccttaataaactaaaacattattgtacataatgtaattataaatctatcagtctgcatggatgcaaactgtgtgtgacaaatcgtcttttaaatggtcaatttcagctgtacatttctcatccaagttgtactctagccattggtttagcgtggacagatactccatctctattatccatttccacagcccaaataaaatatgttaagcaggctcagaatgaatatgaagaattccatttccgtagatgttttctaaaagtcagattgtggaaatttcaaataagtattttcaatttcctccctcactccctcttaccttccccaagacatcaaacacctggcatggtagattatagaaagagacactgagagagagaattaaattttctaatgggaattagaatatctggtttacctccaaattaagtttctttacaggaagttgggaccagctggaaattgtattatcatttcggaacacaatattacttccttagagagagatggatttttgttggcaacataaaagaataagtgatggggtatctatgtgagtaaatttttaaattccaagttaatatgcttgcaaacattactgctgcgttatggcaccaagagttggttgaaattacccacgtagtcatactcctgtgctctgtcaccatggacttcacatcatttgctgcccactagttcaatcgcatttaatcctagcaatatgtaagtttaaaggactgcattcccctctactttaaagcttatatcctagatactcagtttgtgaccttggttgacctttggcatttcacgcaagttgaacagcaaaatcaaagcagccatttttaattcgtcattcccaagccctatctgagtggttccaaagtcacctgagaggaaggtttaaattagtccctctcaccccaccattgctataaaacagcaacgttttcattccaattcagccataaagaggtttctcttcttactgggttttgaaatcatttccttttttctttcgtttgcccttcttccttcctgtgttgtactatctctccaaccacgtctataagcagactcaatcttcttgcagaggaagatgagctggcactctgcaaacttctttaataaggacccaaagtttacttgacattaacctctagtaggtgagaaattccaaaaaagctttggggtacattagaatttccaaagctctgaaagatatttcaggagatttaacagcagagttgcaacttacacgtgaattctggtctatagtggtcatgtgagttaaattcgaatcctctacttgtatgaccctaggaatagattggaatactgcagaggaccaaagctgaggcatgctaaacagctgcttggaggtggaagcaagttcagtcacctactcagcttcctctctccaccacccagttcctccctcagtatcacattatttttttcttctgcttttcattaacctaactcatctcatcagtacaaccattttcttattctctaactacccccaattcctttagcctctccccacctgtccaaactcagctcagccgtgggccaatgcagcttacagacggttgcagagctaggaagaaaacccagctctcccaaccctgatcgtggaggtctctggccccccaacactgccttctgggggctgcattttttttttttttttgagaagaggtcttttgggatgcatggtgctccacatacagcttcacaaaatatttcattataagagaaaccccttgatttttatttctttttcttttgttttctggattacctgccttcagtaagcagatgcagacccacttgtaaggagtctggttagtgatgagaaaaggatgaaatctagatacaaaagtcaccttgaaggtgatgatggatctttaatccacttgactaagtgtctggaagagctacttgctcttccacccctcatctcaaatgagaggagcagaagtttaacttcctcaaatagcccagctctggctaaaacccaaagaagaaaggtcaaaggaagggaaagcatgtcaggggctggtttgtgacttggcaggaccaggaaacagcagccactgacagcccagagaaggtgactaagggctggcagaagattagaatgttaatttggctgctgtccggactaggaggccatgttcaatggcagtcagaatttgtgttctgcgcattgctgggtctataaatatgataagcaagtggtgacagaattatagtataaggtgatgtactacatgcagatcataaaggctttggttttaagacttaagtagaaaatccctctcctagcttattacccaacaatattcagataatgagcttttggagaatatctttttcctatcactagaaagatttacctgggaactgtctaaagtctacacacatatttccaaagcctttaaataccaactgcagcatggagagagaggggtggcagaagctgaaatgcctcaaaagccatttaagtgttacgttgcaggattttcagtccttctcgttatgtaaaagtagataaatatagacgttattctcaacactaccctatagtatcacagtggtccaaatgccagagcttacagataatgtcatcacagtgcctagaaactcgaactgtaatatatcgtagcattttcttggtgtttcttaaagtttctttccacaatacaaggtcctctctgcctcttgtttcttggagagttcaccccactgatgagtcttccttccctgttggactcagtcatttggggaacagtcttagaagcacatatcaaccaaggaagaaacttcctggatatctattgcccacttgcccaggtctaataaacactaaaggggggaaattgaaaggagctgccaactggtcaacgtggaagggcggttccaccctagattggtgtctttctttttcttcctttttttaaaaaaaatctatttcttaaataataataaatgcacatgatgtgttaaatatgtacatatatatttcaaaagaaaaaatggggcacaagattgtcttacaagtcgtgctggctaatttttagtttgtattcataagtggtttttaaaagccttttttaaagtgtaatttgcatgttctactttgattgtatgtaaacatattttagaacaaaaaatgtatttgtattttattgaatatagaggcaagaaaattgtacattgtttgaaatgttctttttgtaacagtttttattcataaagcatttttgtacatttaaaatgaacatggacttgctgttatttgaggcgtagatacatctagcatgcttactgtcatgctcctccatggtcttagatgttgggttttaaacatttttttctaaaagaaagctcagtcttttccgctaccagatcaggttagcacagtatagagcacttaactattaaaaaaaaaaagttaatcctattcatatgttattcattgtgtgaaattaaagacattcaattcagtctaa

SEQ ID NO: 7 - DNA, 20 bp, Homo sapiens
  source 1..20: mol_type = other DNA
aggaggatgaactaggctca

SEQ ID NO: 8 - DNA, 20 bp, Homo sapiens
  source 1..20: mol_type = other DNA
cggaaagaggaaacacaaac

SEQ ID NO: 9 - DNA, 20 bp, Homo sapiens
  source 1..20: mol_type = other DNA
aagaggaaacacaaaccgca

SEQ ID NO: 10 - DNA, 20 bp, Homo sapiens
  source 1..20: mol_type = other DNA
taacaaagatctgctcttgg

SEQ ID NO: 11 - DNA, 20 bp, Homo sapiens
  source 1..20: mol_type = other DNA
caaaggcccagggtaagaaa

SEQ ID NO: 12 - DNA, 20 bp, Homo sapiens
  source 1..20: mol_type = other DNA
ttaaagcggtggtggaaaag

SEQ ID NO: 13 - DNA, 20 bp, Homo sapiens
  source 1..20: mol_type = other DNA
caggatactttccaactctg

SEQ ID NO: 14 - DNA, 20 bp, Homo sapiens
  source 1..20: mol_type = other DNA
atggagtccagggaaacctt

SEQ ID NO: 15 - DNA, 25 bp, Homo sapiens
  source 1..25: mol_type = other DNA
gaaagaggaaacacaaaccgcaggc

SEQ ID NO: 16 - DNA, 20 bp, Homo sapiens
  source 1..20: mol_type = other DNA
cacaaaccgcaggcccccgc

SEQ ID NO: 17 - DNA, 21 bp, Homo sapiens
  source 1..21: mol_type = other DNA
aggcggaaagaggaaacacaa

SEQ ID NO: 18 - DNA, 20 bp, Homo sapiens
  source 1..20: mol_type = other DNA
atactttccaactctgaggg

SEQ ID NO: 19 - DNA, 20 bp, Homo sapiens
  source 1..20: mol_type = other DNA
ttaacaggatactttccaac

SEQ ID NO: 20 - DNA, 9909 bp, Homo sapiens
  source 1..9909: mol_type = other DNA
gtaacttgcaggttaagtggagctagagagctacattgtaacctagttggatttttttggggggggaatgtatcagaatctaacgtgtcgttaatagaaagaagaacaggcaagaagtggtccagccggcgaagttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacaggcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctgaggggaataagaaggatccccgtgtccctaagttgagtaaaatgatagagaatgagtccccctcagttggccttgaaactggtggaaatgctgagaaagttatcccaggaggtgtgtctaagccgcggaagccacccatggtcatgacacctccaacgtgcacagatcactctccatccagaaagctgccagaaatccagcatccaaaatttgctgcaaaacgaaggtggacttgcagcaaaccaaaacctagcaccatgcttcgagaggcagttatggccacctctgataaactgatgctggagcccccgtctgcatatcccatcaccccatccagccctctctacaccaacacagacagtcttactgtgatcactccagtcaaaaagaagcggggacgaccaaagaagcagcctttgctcacagtcgagacgattcatgagggaacttccaccagccccgtcagtcccatcagccgagagtttcctggcactaagaaaagaaagcgacgacgcaatttagcgaagttggcccagctagtgccgggagaggacaaacccatgagcgagatgaaatttcacaagaaagttggaaagctcggcgtgttggataagaagaccatcaaaactatcaataagatgaagacactcaagaggaaaaacatcttgaatcagatcttgtcctgttccagcagcgttgctctgaaggcaaaagctcccccagagaccagccctggggcagcagccattgaaagcaaactgggcaagcagattaatgtcagcaagaggggaaccatctacattggcaagaagcggggcaggaagccaagagcagagctgccacccccatccgaagaacccaaaacagccatcaagcaccccaggcctgtttctagccagccggatgttccagccgtgccttccaactttcagtcacttgtggcgtcttcaccagcagctatgcacccactttcaacacagttaggtgggtccaatggcaacctgagccctgccagcactgaaaccaatttttcagagttgaaaactatgccaaatctccagcccatcagtgctcttccaaccaaaacccaaaagggaatacacagtggaacctggaagctgtctccacccagactgatggccaactccccttcacacctgtgcgagattggctccctaaaggaaatcacgctgtcccctgtgagcgagtcccacagtgaggagacgatccccagcgacagcggcattgggacagacaacaacagcacttctgaccaagcggagaagagctcagaatcccgaaggaggtactcttttgatttctgctccctggacaacccggaggccattccgtccgacaccagcacaaagaaccggcatggccaccggcaaaagcatctcattgtggacaactttctggcccacgaaagcctcaagaagccaaagcacaagaggaaacggaaaagcctgcaaaaccgcgatgacctccagtttctggcagacctggaggagctaatcaccaagttccaagtgttcagaatctcccaccggagttacaccttctaccacgagaatccatatcccagcatttttcggattaattttgatcactattacccggtgccatatatccagtatgacccgttgctctatcttcgtaggacttcagacttgaagtcaaagaagaagcgtggtaggcctgcaaaaaccaatgacaccatgacaaaggtgccttttttacaagggttcagctaccctattcccagtggaagttactatgcaccctatggaatgccttacacatcaatgcctatgatgaaccttggttattacggtcagtacccagctcctttgtacctatcgcacacgcttggagcagcttccccattcatgaggccaacagtgccaccacctcagttccacacaaactcccacgtaaagatgtccggtgcagctaagcataaagccaagcatggagtacacctgcagggacctgttagcatgggccttggtgacatgcagccttctctgaaccctcccaaggtaggcagtgccagtctgtccagtggtcggctccataagaggaaacacaaacacaagcataagcacaaggaagaccggatcctagggacccatgacaacctgagtggtctttttgcaggcaaagccacaggcttctccagccacatcctgagcgagcggctgagtagcgcagacaaagagctcccgctggtgagtgagaagaacaagcataaggagaaacagaagcaccagcacagcgaagccggccacaaagcttctaagaacaactttgaggtggacaccctgtctacactgtcactttccgacgcccagcattggacacaggccaaggaaaaaggagacttgagcagtgagcctgtggactcatgcacgaaaagatactctggcagtggcggggatggtggcagcacgagatcagagaacctggacgtgttcagtgaaatgaacccttcgaatgacaagtgggacagtgacgtgagtgggagtaaaaggaggagctatgaaggctttggaacgtacagggaaaaggacatccaagccttcaagatgaaccgcaaggagagaagttcttatgactcctccatgtctccagggatgccaagtccccacttaaaagtggaccagacagcagtgcatagtaagaacgaaggctcagtgcccaccatgatgaccaggaagaagccagccgcagttgacagtgttacaattccaccagccccagtgttatctctccttgctgcatctgcagcaacgtcggatgcagtcggctcctccctgaagaagaggttcaagcggcgggagatcgaagccatccagtgcgaagtgcggaagatgtgcaactacaccaagatcctgtccaccaagaagaacctggaccacgtgaacaagatcctgaaggccaagcggctgcagagacaatcaaaaacaggcaacaacttcgtgaagaagaggcgcgggcgtcccaggaagcagcccacccagttcgatgaggactccagagaccaaatgccggtgctggaaaaatgcatcgacctgcccagcaaaagaggccagaagcccagcctgagcccgctggtgctggagcccgccgccagccaagacaccatcatggccaccatcgaggcggtcatccacatggcccgggaggcgccgcccctgcccccgccaccgccgccgcccctgccgccaccgccgccaccacccctgcccccgccaccccctctacccaagaccccccgaggcggaaagaggaaacacaaaccgcaggcccccgctcagcccccacagcagtcgcccccgcagcagccccttccccaggaagaggaggtgaaagccaaaaggcagaggaagtcccgagggagtgagagcgaggtccttccctagggcgggtctgggcgtctgcacctggggcctagggaactgacacgtgggaagcgcagtgagccggggcgggggcggaatcccccgctgcagggacacccacgcccttctctccagaagccgggcaggcagaatccggccagacgacggggctgagccatcaggagctcttgggaaagcaaagcagggagacaccttcagaagaagcttgtctgagcttcaccgcagctcccaccacgcggcgcttcagtacggctggatcctccgcaggcgagcggaaggcccccaggaggagcaggctggtggcactctcctgacctcacgctgccaaggtgcgccctcgactcccgatttcatttgctggccaggaaaatcgaaagggaaacaccctcaattaggaaacattccaaggggagaaaagctgcaaattgctcaactggcattgctgtaacccgttccatttaattggtttttatttcttttgattttcaagctctgctaagctttggggtaccaacgtcatcttcaggtgacctgaatctttcccttaaccgtacagtttctcgatggaattgtgtgatcagaaggtggaattctagtgataggcgacctcagacccgcattcatgttctgtgtgcctcttctattgcacatacactgatttttagcattgtctattcctatttttcctttgcccattgtacttccatatatcttttcattaacttacttgctgccttttttttttcttggtacacatttaaataaagtaatccttaacctgtgctgtaaagttcacccttggcatgctgttccaagaacctgggtttgaatcccaatcgttgtgaaacatactcagtattgataaaacctttttaataagtgatgcagagcagccaaggatatgttgacccagatgtcaaccaggctatttttatacttaaaacatgtcagcagagcataggcagaataaaatggtttaaataccccacagcaaatagagtaactgacaaaccaccaaaaactgaaaccccagacccaccagaaagacaagtgtctagcaatgccttggtacctgatctttttcattcaaataattgtcataggttgttcaaaaaaaaaaaaaaaaaggaaacaaaaacacaatagaagtccattatcccttgataattatttcttaaaagcaaatgggagtaagtgttcttatctggaaaaataaataaataaaatgctcaaagggtatcaccacttcaattttatcaagccaccttggacaaagtatccaaattgtggttgccttaataaactaaaacattattgtacataatgtaattataaatctatcagtctgcatggatgcaaactgtgtgtgacaaatcgtcttttaaatggtcaatttcagctgtacatttctcatccaagttgtactctagccattggtttagcgtggacagatactccatctctattatccatttccacagcccaaataaaatatgttaagcaggctcagaatgaatatgaagaattccatttccgtagatgttttctaaaagtcagattgtggaaatttcaaataagtattttcaatttcctccctcactccctcttaccttccccaagacatcaaacacctggcatggtagattatagaaagagacactgagagagagaattaaattttctaatgggaattagaatatctggtttacctccaaattaagtttctttacaggaagttgggaccagctggaaattgtattatcatttcggaacacaatattacttccttagagagagatggatttttgttggcaacataaaagaataagtgatggggtatctatgtgagtaaatttttaaattccaagttaatatgcttgcaaacattactgctgcgttatggcaccaagagttggttgaaattacccacgtagtcatactcctgtgctctgtcaccatggacttcacatcatttgctgcccactagttcaatcgcatttaatcctagcaatatgtaagtttaaaggactgcattcccctctactttaaagcttatatcctagatactcagtttgtgaccttggttgacctttggcatttcacgcaagttgaacagcaaaatcaaagcagccatttttaattcgtcattcccaagccctatctgagtggttccaaagtcacctgagaggaaggtttaaattagtccctctcaccccaccattgctataaaacagcaacgttttcattccaattcagccataaagaggtttctcttcttactgggttttgaaatcatttccttttttctttcgtttgcccttcttccttcctgtgttgtactatctctccaaccacgtctataagcagactcaatcttcttgcagaggaagatgagctggcactctgcaaacttctttaataaggacccaaagtttacttgacattaacctctagtaggtgagaaattccaaaaaagctttggggtacattagaatttccaaagctctgaaagatatttcaggagatttaacagcagagttgcaacttacacgtgaattctggtctatagtggtcatgtgagttaaattcgaatcctctacttgtatgaccctaggaatagattggaatactgcagaggaccaaagctgaggcatgctaaacagctgcttggaggtggaagcaagttcagtcacctactcagcttcctctctccaccacccagttcctccctcagtatcacattatttttttcttctgcttttcattaacctaactcatctcatcagtacaaccattttcttattctctaactacccccaattcctttagcctctccccacctgtccaaactcagctcagccgtgggccaatgcagcttacagacggttgcagagctaggaagaaaacccagctctcccaaccctgatcgtggaggtctctggccccccaacactgccttctgggggctgcattttttttttttttttgagaagaggtcttttgggatgcatggtgctccacatacagcttcacaaaatatttcattataagagaaaccccttgatttttatttctttttcttttgttttctggattacctgccttcagtaagcagatgcagacccacttgtaaggagtctggttagtgatgagaaaaggatgaaatctagatacaaaagtcaccttgaaggtgatgatggatctttaatccacttgactaagtgtctggaagagctacttgctcttccacccctcatctcaaatgagaggagcagaagtttaacttcctcaaatagcccagctctggctaaaacccaaagaagaaaggtcaaaggaagggaaagcatgtcaggggctggtttgtgacttggcaggaccaggaaacagcagccactgacagcccagagaaggtgactaagggctggcagaagattagaatgttaatttggctgctgtccggactaggaggccatgttcaatggcagtcagaatttgtgttctgcgcattgctgggtctataaatatgataagcaagtggtgacagaattatagtataaggtgatgtactacatgcagatcataaaggctttggttttaagacttaagtagaaaatccctctcctagcttattacccaacaatattcagataatgagcttttggagaatatctttttcctatcactagaaagatttacctgggaactgtctaaagtctacacacatatttccaaagcctttaaataccaactgcagcatggagagagaggggtggcagaagctgaaatgcctcaaaagccatttaagtgttacgttgcaggattttcagtccttctcgttatgtaaaagtagataaatatagacgttattctcaacactaccctatagtatcacagtggtccaaatgccagagcttacagataatgtcatcacagtgcctagaaactcgaactgtaatatatcgtagcattttcttggtgtttcttaaagtttctttccacaatacaaggtcctctctgcctcttgtttcttggagagttcaccccactgatgagtcttccttccctgttggactcagtcatttggggaacagtcttagaagcacatatcaaccaaggaagaaacttcctggatatctattgcccacttgcccaggtctaataaacactaaaggggggaaattgaaaggagctgccaactggtcaacgtggaagggcggttccaccctagattggtgtctttctttttcttcctttttttaaaaaaaatctatttcttaaataataataaatgcacatgatgtgttaaatatgtacatatatatttcaaaagaaaaaatggggcacaagattgtcttacaagtcgtgctggctaatttttagtttgtattcataagtggtttttaaaagccttttttaaagtgtaatttgcatgttctactttgattgtatgtaaacatattttagaacaaaaaatgtatttgtattttattgaatatagaggcaagaaaattgtacattgtttgaaatgttctttttgtaacagtttttattcataaagcatttttgtacatttaaaatgaacatggacttgctgttatttgaggcgtagatacatctagcatgcttactgtcatgctcctccatggtcttagatgttgggttttaaacatttttttctaaaagaaagctcagtcttttccgctaccagatcaggttagcacagtatagagcacttaactattaaaaaaaaaaagttaatcctattcatatgttattcattgtgtgaaattaaagacattcaattcagtctaa

SEQ ID NO: 21 - DNA, 4791 bp, Homo sapiens
  source 1..4791: mol_type = other DNA
atggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacaggcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctgaggggaataagaaggatccccgtgtccctaagttgagtaaaatgatagagaatgagtccccctcagttggccttgaaactggtggaaatgctgagaaagttatcccaggaggtgtgtctaagccgcggaagccacccatggtcatgacacctccaacgtgcacagatcactctccatccagaaagctgccagaaatccagcatccaaaatttgctgcaaaacgaaggtggacttgcagcaaaccaaaacctagcaccatgcttcgagaggcagttatggccacctctgataaactgatgctggagcccccgtctgcatatcccatcaccccatccagccctctctacaccaacacagacagtcttactgtgatcactccagtcaaaaagaagcggggacgaccaaagaagcagcctttgctcacagtcgagacgattcatgagggaacttccaccagccccgtcagtcccatcagccgagagtttcctggcactaagaaaagaaagcgacgacgcaatttagcgaagttggcccagctagtgccgggagaggacaaacccatgagcgagatgaaatttcacaagaaagttggaaagctcggcgtgttggataagaagaccatcaaaactatcaataagatgaagacactcaagaggaaaaacatcttgaatcagatcttgtcctgttccagcagcgttgctctgaaggcaaaagctcccccagagaccagccctggggcagcagccattgaaagcaaactgggcaagcagattaatgtcagcaagaggggaaccatctacattggcaagaagcggggcaggaagccaagagcagagctgccacccccatccgaagaacccaaaacagccatcaagcaccccaggcctgtttctagccagccggatgttccagccgtgccttccaactttcagtcacttgtggcgtcttcaccagcagctatgcacccactttcaacacagttaggtgggtccaatggcaacctgagccctgccagcactgaaaccaatttttcagagttgaaaactatgccaaatctccagcccatcagtgctcttccaaccaaaacccaaaagggaatacacagtggaacctggaagctgtctccacccagactgatggccaactccccttcacacctgtgcgagattggctccctaaaggaaatcacgctgtcccctgtgagcgagtcccacagtgaggagacgatccccagcgacagcggcattgggacagacaacaacagcacttctgaccaagcggagaagagctcagaatcccgaaggaggtactcttttgatttctgctccctggacaacccggaggccattccgtccgacaccagcacaaagaaccggcatggccaccggcaaaagcatctcattgtggacaactttctggcccacgaaagcctcaagaagccaaagcacaagaggaaacggaaaagcctgcaaaaccgcgatgacctccagtttctggcagacctggaggagctaatcaccaagttccaagtgttcagaatctcccaccggagttacaccttctaccacgagaatccatatcccagcatttttcggattaattttgatcactattacccggtgccatatatccagtatgacccgttgctctatcttcgtaggacttcagacttgaagtcaaagaagaagcgtggtaggcctgcaaaaaccaatgacaccatgacaaaggtgccttttttacaagggttcagctaccctattcccagtggaagttactatgcaccctatggaatgccttacacatcaatgcctatgatgaaccttggttattacggtcagtacccagctcctttgtacctatcgcacacgcttggagcagcttccccattcatgaggccaacagtgccaccacctcagttccacacaaactcccacgtaaagatgtccggtgcagctaagcataaagccaagcatggagtacacctgcagggacctgttagcatgggccttggtgacatgcagccttctctgaaccctcccaaggtaggcagtgccagtctgtccagtggtcggctccataagaggaaacacaaacacaagcataagcacaaggaagaccggatcctagggacccatgacaacctgagtggtctttttgcaggcaaagccacaggcttctccagccacatcctgagcgagcggctgagtagcgcagacaaagagctcccgctggtgagtgagaagaacaagcataaggagaaacagaagcaccagcacagcgaagccggccacaaagcttctaagaacaactttgaggtggacaccctgtctacactgtcactttccgacgcccagcattggacacaggccaaggaaaaaggagacttgagcagtgagcctgtggactcatgcacgaaaagatactctggcagtggcggggatggtggcagcacgagatcagagaacctggacgtgttcagtgaaatgaacccttcgaatgacaagtgggacagtgacgtgagtgggagtaaaaggaggagctatgaaggctttggaacgtacagggaaaaggacatccaagccttcaagatgaaccgcaaggagagaagttcttatgactcctccatgtctccagggatgccaagtccccacttaaaagtggaccagacagcagtgcatagtaagaacgaaggctcagtgcccaccatgatgaccaggaagaagccagccgcagttgacagtgttacaattccaccagccccagtgttatctctccttgctgcatctgcagcaacgtcggatgcagtcggctcctccctgaagaagaggttcaagcggcgggagatcgaagccatccagtgcgaagtgcggaagatgtgcaactacaccaagatcctgtccaccaagaagaacctggaccacgtgaacaagatcctgaaggccaagcggctgcagagacaatcaaaaacaggcaacaacttcgtgaagaagaggcgcgggcgtcccaggaagcagcccacccagttcgatgaggactccagagaccaaatgccggtgctggaaaaatgcatcgacctgcccagcaaaagaggccagaagcccagcctgagcccgctggtgctggagcccgccgccagccaagacaccatcatggccaccatcgaggcggtcatccacatggcccgggaggcgccgcccctgcccccgccaccgccgccgcccctgccgccaccgccgccaccacccctgcccccgccaccccctctacccaagaccccccgaggcggaaagaggaaacacaaaccgcaggcccccgctcagcccccacagcagtcgcccccgcagcagccccttccccaggaagaggaggtgaaagccaaaaggcagaggaagtcccgagggagtgagagcgaggtccttccctag

SEQ ID NO: 22 - protein, 1596 aa, Homo sapiens
MESRETLSSSRQRGGESDFLPVSSAKPPAAPGCAGEPLLSTPGPGKGIPVGGERMEPEEEDELGSGRDVDSNSNADSEKWVAGDGLEEQEFSIKEANFTEGSLKLKIQTTKRAKKPPKNLENYICPPEIKITIKQSGDQKVSRAGKNSKATKEEERSHSKKKLLTASDLAASDLKGFQPQAYERPQKHSTLHYDTGLPQDFTGDTLKPKHQQKSSSQNHMDWSTNSDSGPVTQNCFISPESGRETASTSKIPALEPVASFAKAQGKKGSAGNTWSQLSNNNKDLLLGGVAPSPSSHSSPAPPSSSAECNGLQPLVDQDGGGTKEPPEPPTVGSKKKSSKKDVISQTIPNPDLDWVKNAQKAFDNTEGKREGYSADSAQEASPARQNVSSASNPENDSSHVRITIPIKAPSLDPTNHKRKKRQSIKAVVEKIMPEKALASGITMSSEVVNRILSNSEGNKKDPRVPKLSKMIENESPSVGLETGGNAEKVIPGGVSKPRKPPMVMTPPTCTDHSPSRKLPEIQHPKFAAKRRWTCSKPKPSTMLREAVMATSDKLMLEPPSAYPITPSSPLYTNTDSLTVITPVKKKRGRPKKQPLLTVETIHEGTSTSPVSPISREFPGTKKRKRRRNLAKLAQLVPGEDKPMSEMKFHKKVGKLGVLDKKTIKTINKMKTLKRKNILNQILSCSSSVALKAKAPPETSPGAAAIESKLGKQINVSKRGTIYIGKKRGRKPRAELPPPSEEPKTAIKHPRPVSSQPDVPAVPSNFQSLVASSPAAMHPLSTQLGGSNGNLSPASTETNFSELKTMPNLQPISALPTKTQKGIHSGTWKLSPPRLMANSPSHLCEIGSLKEITLSPVSESHSEETIPSDSGIGTDNNSTSDQAEKSSESRRRYSFDFCSLDNPEAIPSDTSTKNRHGHRQKHLIVDNFLAHESLKKPKHKRKRKSLQNRDDLQFLADLEELITKFQVFRISHRSYTFYHENPYPSIFRINFDHYYPVPYIQYDPLLYLRRTSDLKSKKKRGRPAKTNDTMTKVPFLQGFSYPIPSGSYYAPYGMPYTSMPMMNLGYYGQYPAPLYLSHTLGAASPFMRPTVPPPQFHTNSHVKMSGAAKHKAKHGVHLQGPVSMGLGDMQPSLNPPKVGSASLSSGRLHKRKHKHKHKHKEDRILGTHDNLSGLFAGKATGFSSHILSERLSSADKELPLVSEKNKHKEKQKHQHSEAGHKASKNNFEVDTLSTLSLSDAQHWTQAKEKGDLSSEPVDSCTKRYSGSGGDGGSTRSENLDVFSEMNPSNDKWDSDVSGSKRRSYEGFGTYREKDIQAFKMNRKERSSYDSSMSPGMPSPHLKVDQTAVHSKNEGSVPTMMTRKKPAAVDSVTIPPAPVLSLLAASAATSDAVGSSLKKRFKRREIEAIQCEVRKMCNYTKILSTKKNLDHVNKILKAKRLQRQSKTGNNFVKKRRGRPRKQPTQFDEDSRDQMPVLEKCIDLPSKRGQKPSLSPLVLEPAASQDTIMATIEAVIHMAREAPPLPPPPPPPLPPPPPPPLPPPPPLPKTPRGGKRKHKPQAPAQPPQQSPPQQPLPQEEEVKAKRQRKSRGSESEVLP

SEQ ID NO: 23 - DNA, 20 bp, synthetic construct
  source 1..20: mol_type = other DNA
gtccacctgagatcaagatc

SEQ ID NO: 24 - DNA, 20 bp, synthetic construct
  source 1..20: mol_type = other DNA
gtccatgtggttctggctgc

SEQ ID NO: 25 - DNA, 20 bp, synthetic construct
  source 1..20: mol_type = other DNA
gctctctgctcctcctgttc

SEQ ID NO: 26 - DNA, 20 bp, synthetic construct
  source 1..20: mol_type = other DNA
cgaccaaatccgttgactcc

SEQ ID NO: 27 - DNA, 20 bp, synthetic construct
  source 1..20: mol_type = other DNA
tgagcctagttcatcctcct

SEQ ID NO: 28 - DNA, 20 bp, synthetic construct
  source 1..20: mol_type = other DNA
gtttgtgtttcctctttccg

SEQ ID NO: 29 - DNA, 20 bp, synthetic construct
  source 1..20: mol_type = other DNA
tgcggtttgtgtttcctctt

SEQ ID NO: 30 - DNA, 20 bp, synthetic construct
  source 1..20: mol_type = other DNA
ccaagagcagatctttgtta

SEQ ID NO: 31 - DNA, 20 bp, synthetic construct
  source 1..20: mol_type = other DNA
tttcttaccctgggcctttg

SEQ ID NO: 32 - DNA, 20 bp, synthetic construct
  source 1..20: mol_type = other DNA
cttttccaccaccgctttaa

SEQ ID NO: 33 - DNA, 20 bp, synthetic construct
  source 1..20: mol_type = other DNA
cagagttggaaagtatcctg

SEQ ID NO: 34 - DNA, 20 bp, synthetic construct
  source 1..20: mol_type = other DNA
aaggtttccctggactccat

SEQ ID NO: 35 - DNA, 25 bp, synthetic construct
  source 1..25: mol_type = other DNA
gcctgcggtttgtgtttcctctttc

SEQ ID NO: 36 - DNA, 20 bp, synthetic construct
  source 1..20: mol_type = other DNA
gcgggggcctgcggtttgtg

SEQ ID NO: 37 - DNA, 21 bp, synthetic construct
  source 1..21: mol_type = other DNA
ttgtgtttcctctttccgcct

SEQ ID NO: 38 - DNA, 20 bp, synthetic construct
  source 1..20: mol_type = other DNA
ccctcagagttggaaagtat

SEQ ID NO: 39 - DNA, 20 bp, synthetic construct
  source 1..20: mol_type = other DNA
gttggaaagtatcctgttaa

SEQ ID NO: 40 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
tgagcctagttcatcctcct

SEQ ID NO: 41 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
gtttgtgtttcctctttccg

SEQ ID NO: 42 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
tgcggtttgtgtttcctctt

SEQ ID NO: 43 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
ccaagagcagatctttgtta

SEQ ID NO: 44 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
tttcttaccctgggcctttg

SEQ ID NO: 45 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
cttttccaccaccgctttaa

SEQ ID NO: 46 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
cagagttggaaagtatcctg

SEQ ID NO: 47 - DNA, 20 bp, synthetic construct
  source 1..20: mol_type = other DNA
  misc_feature 1..5: standard_name = Positions 1-5 are connected by phosphorothioate linkages
  misc_feature 16..20: standard_name = Positions 16-20 are connected by phosphorothioate linkages
aaggtttccctggactccat

SEQ ID NO: 48 - DNA, 20 bp, synthetic construct
  source 1..20: mol_type = other DNA
  misc_feature 1..20: standard_name = Positions 1-20 are connected by phosphorothioate linkages
gtgcaacagcacctggaagg

SEQ ID NO: 49 - DNA, 20 bp, synthetic construct
  source 1..20: mol_type = other DNA
  misc_feature 1..5: standard_name = Each of positions 1-5 is a locked nucleic acid base
  misc_feature 16..20: standard_name = Each of positions 16-20 is a locked nucleic acid base
  misc_feature 5..15: standard_name = Positions 5-15 are connected by phosphorothioate linkages
tgcggtttgtgtttcctctt

SEQ ID NO: 50 - DNA, 20 bp, synthetic construct
  source 1..20: mol_type = other DNA
  misc_feature 1..5: standard_name = Each of positions 1-5 is a locked nucleic acid base
  misc_feature 16..20: standard_name = Each of positions 16-20 is a locked nucleic acid base
  misc_feature 5..15: standard_name = Positions 5-15 are connected by phosphorothioate linkages
tctctgctgtggttgttctt

SEQ ID NO: 51 - DNA, 20 bp, synthetic construct
  source 1..20: mol_type = other DNA
  misc_difference 5..16: standard_name = Positions 5-16 are connected by phosphorothioate linkages
  misc_feature 1..5: standard_name = Each of positions 1-5 is a 2'-MOE RNA base
  misc_feature 16..20: standard_name = Each of positions 16-20 is a 2'-MOE RNA base
  misc_feature 1: standard_name = Position 1 has a FAM tag
tgcggtttgtgtttcctctt

SEQ ID NO: 52 - DNA, 25 bp, synthetic construct
  source 1..25: mol_type = other DNA
  misc_feature 6..7: standard_name = Positions 6-7 are connected by phosphorothioate linkages
  misc_feature 8..9: standard_name = Positions 8-9 are connected by phosphorothioate linkages
  misc_feature 10..15: standard_name = Positions 10-15 are connected by phosphorothioate linkages
  misc_feature 16..17: standard_name = Positions 16-17 are connected by phosphorothioate linkages
  misc_feature 19..20: standard_name = Positions 19-20 are connected by phosphorothioate linkages
  misc_feature 1..5: standard_name = Each of positions 1-5 is a 2'-MOE RNA base
  misc_feature 21..25: standard_name = Each of positions 21-25 is a 2'-MOE RNA base
gcctgcggtttgtgtttcctctttc

SEQ ID NO: 53 - DNA, 20 bp, synthetic construct
  source 1..20: mol_type = other DNA
  misc_feature 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
gcgggggcctgcggtttgtg

SEQ ID NO: 54 - DNA, 21 bp, synthetic construct
  source 1..21: mol_type = other DNA
  misc_feature 1..5: standard_name = Each of positions 1-5 is a 2'-MOE RNA base
  misc_feature 17..21: standard_name = Each of positions 17-21 is a 2'-MOE RNA base
  misc_feature 6..17: standard_name = Positions 6-17 are connected by phosphorothioate linkages
ttgtgtttcctctttccgcct

SEQ ID NO: 55 - DNA, 20 bp, synthetic construct
  source 1..20: mol_type = other DNA
  misc_feature 1..5: standard_name = Each of positions 1-5 is a 2'-MOE RNA base
  misc_difference 16..20: standard_name = Each of positions 16-20 is a 2'-MOE RNA base
  misc_feature 6..16: standard_name = Positions 6-16 are connected by phosphorothioate linkages
tgcggtttgtgtttcctctt

SEQ ID NO: 56 - DNA, 20 bp, synthetic construct
  source 1..20: mol_type = other DNA
  misc_feature 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
tttgttgcgttgtctcttgc

SEQ ID NO: 57 - DNA, 20 bp, synthetic construct
  source 1..20: mol_type = other DNA
  misc_feature 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
ccctcagagttggaaagtat

SEQ ID NO: 58 - DNA, 20 bp, synthetic construct
  source 1..20: mol_type = other DNA
  misc_difference 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
gttggaaagtatcctgttaa

SEQ ID NO: 59 - DNA, 20 bp, synthetic construct
  source 1..20: mol_type = other DNA
  misc_difference 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
cagagttggaaagtatcctg

SEQ ID NO: 60 - DNA, 20 bp, synthetic construct
  source 1..20: mol_type = other DNA
  misc_feature 1..5: standard_name = Each of positions 1-5 is a 2'-MOE RNA base
  misc_feature 16..20: standard_name = Each of positions 16-20 is a 2'-MOE RNA base
  misc_feature 6..16: standard_name = Positions 6-16 are connected by phosphorothioate linkages
gctatattaggcgatacgag

SEQ ID NO: 61 - DNA, 636 bp, Homo sapiens
  source 1..636: mol_type = other DNA
gaaagcaaactgggcaagcagattaatgtcagcaagaggggaaccatctacattggcaagaagcggggcaggaagccaagagcagagctgccacccccatccgaagaacccaaaacagccatcaagcaccccaggcctgtttctagccagccggatgttccagccgtgccttccaactttcagtcacttgtggcgtcttcaccagcagctatgcacccactttcaacacagttaggtgggtccaatggcaacctgagccctgccagcactgaaaccaatttttcagagttgaaaactatgccaaatctccagcccatcagtgctcttccaaccaaaacccaaaagggaatacacagtggaacctggaagctgtctccacccagactgatggccaactccccttcacacctgtgcgagattggctccctaaaggaaatcacgctgtcccctgtgagcgagtcccacagtgaggagacgatccccagcgacagcggcattgggacagacaacaacagcacttctgaccaagcggagaagagctcagaatcccgaaggaggtactcttttgatttctgctccctggacaacccggaggccattccgtccgacaccagcacaaagaaccggcatggccac

SEQ ID NO: 62 - protein, 212 aa, Homo sapiens
ESKLGKQINVSKRGTIYIGKKRGRKPRAELPPPSEEPKTAIKHPRPVSSQPDVPAVPSNFQSLVASSPAAMHPLSTQLGGSNGNLSPASTETNFSELKTMPNLQPISALPTKTQKGIHSGTWKLSPPRLMANSPSHLCEIGSLKEITLSPVSESHSEETIPSDSGIGTDNNSTSDQAEKSSESRRRYSFDFCSLDNPEAIPSDTSTKNRHGH

SEQ ID NO: 63 - DNA, 1366 bp, Homo sapiens
  source 1..1366: mol_type = other DNA
atggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacaggcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctg

SEQ ID NO: 64 - DNA, 1370 bp, Homo sapiens
  source 1..1370: mol_type = other DNA
atggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacaggcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctgaggg

SEQ ID NO: 65 - DNA, 859 bp, Homo sapiens
  source 1..859: mol_type = other DNA
atggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacaggcttacgagaggccccagaaacattcaactctccattatgacacgggcctcccacaggacttcaccggtgacaccttaaaaccaaagcaccagcaaaaaagcagcagccagaaccacatggactggtccaccaactctgacagcggacccgtcactcagaattgcttcatcagtccagagtctggcagagaaactgcaagcaccagcaagatccccgctcttgagcccgtggcttcctttgcaaaggcccagggtaagaaaggcagtgcagggaacacgtggagtcagttgtctaacaataacaaagatctgctcttgg

SEQ ID NO: 66 - DNA, 585 bp, Homo sapiens
  source 1..585: mol_type = other DNA
caaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctg

SEQ ID NO: 67 - DNA, 589 bp, Homo sapiens
  source 1..589: mol_type = other DNA
caaaggcccagggtaagaaaggcagtgcagggaacacgtggagtcagttgtctaacaataacaaagatctgctcttgggaggtgtggctccatccccaagcagccacagctcaccagccccacccagcagctctgctgagtgcaacgggcttcagcccttggtggatcaagatggaggaggtacaaaggagcccccagaaccacctacggtgggcagcaagaaaaagtccagtaaaaaagatgtgataagtcagaccataccaaacccagacctggattgggtcaagaatgcccagaaagcatttgacaatacagaagggaaaagggaaggttattccgcagatagtgcccaagaggcatcaccagccaggcagaacgtgagttctgccagtaatcctgaaaatgactcaagtcatgtccggattactatccccatcaaggcaccctctctggatccaaccaaccataagaggaaaaaaagacagtccattaaagcggtggtggaaaagatcatgccagagaaagccttggcttctggaatcaccatgagcagtgaagtagttaacaggatactttccaactctgaggg

SEQ ID NO: 68 - DNA, 189 bp, Homo sapiens
  source 1..189: mol_type = other DNA
atggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaacta

SEQ ID NO: 69 - DNA, 78 bp, Homo sapiens
  source 1..78: mol_type = other DNA
caaaggcccagggtaagaaaggcagtgcagggaacacgtggagtcagttgtctaacaataacaaagatctgctcttgg

SEQ ID NO: 70 - DNA, 96 bp, Homo sapiens
  source 1..96: mol_type = other DNA
ttaaagcggtggtggaaaagatcatgccagagaaagccttggcttctggaatcaccatgagcagtgaagtagttaacaggatactttccaactctg

SEQ ID NO: 71 - DNA, 100 bp, Homo sapiens
  source 1..100: mol_type = other DNA
ttaaagcggtggtggaaaagatcatgccagagaaagccttggcttctggaatcaccatgagcagtgaagtagttaacaggatactttccaactctgaggg

SEQ ID NO: 72 - DNA, 31 bp, Homo sapiens
  source 1..31: mol_type = other DNA
tagttaacaggatactttccaactctgaggg

SEQ ID NO: 73 - DNA, 27 bp, Homo sapiens
  source 1..27: mol_type = other DNA
aggcggaaagaggaaacacaaaccgca

SEQ ID NO: 74 - DNA, 36 bp, Homo sapiens
  source 1..36: mol_type = other DNA
aggcggaaagaggaaacacaaaccgcaggcccccgc

SEQ ID NO: 75 - protein, 7 aa, Homo sapiens
SDSGIGT

SEQ ID NO: 76 - protein, 7 aa, Homo sapiens
RDSSSGT